(19) United States
(12) Patent Application Publication
(10) Pub. No.: US 2023/0393143 A1
(43) Pub. Date: Dec. 7, 2023

(54) SYSTEMS AND METHODS FOR ALLERGEN DETECTION

(71) Applicant: SiPhox, Inc., Burlington, MA (US)

(72) Inventors: Michael Dubrovsky, Somerville, MA (US); Eric Hsu, Austin, TX (US); Elaine Taine, Columbia, SC (US); Xabier Arias, San Sebastian (ES); Yulia Rybakova, Arlington, MA (US)

(73) Assignee: SiPhox, Inc., Burlington, MA (US)

(21) Appl. No.: 18/199,286

(22) Filed: May 18, 2023

Related U.S. Application Data
(60) Provisional application No. 63/343,276, filed on May 18, 2022.

(51) Int. Cl. G01N 33/68

(52) U.S. Cl. CPC G01N 33/6854; G01N 2333/415; G01N 2333/435; G01N 2800/24

(57) ABSTRACT

Disclosed herein are methods of allergen testing and IgG depletion. In some embodiments, testing for IgE antibodies specific for an allergen may be improved by first depleting IgG in a sample. In some embodiments, IgG may be depleted using Biotinylated Protein G (bPG) and streptavidin (SA).

### CROSS-REFERENCE TO RELATED APPLICATIONS

This application claims the benefit of priority of U.S. Provisional Patent Application Ser. No. 63/343,276, filed on May 18, 2022, and titled “Specific IgG depletion using Protein G and Streptavidin,” which is incorporated by reference herein in its entirety.

### FIELD OF THE INVENTION

The present invention generally relates to the field of allergen testing. In particular, the present invention is directed to systems and methods for allergen detection.

### BACKGROUND

The increase in allergy diagnoses in both adults and children is occurring at a staggering rate in the US. However, blood-based anti-allergen specific IgE tests may be made less reliable by the presence of IgG.

### SUMMARY OF THE DISCLOSURE

In an aspect, a method of allergen testing includes obtaining a subject sample; depleting IgG in the subject sample; and determining a level of IgE specific to an allergen in the subject sample.

In another aspect, a method of IgG depletion includes obtaining a sample; adding an IgG binder to the sample; and adding an IgG binder aggregator to the sample.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 depicts an exemplary principle for an allergen detection assay;

FIG. 2 depicts an exemplary mechanism by which sIgG may interfere with the assay of FIG. 1;

FIG. 3 depicts an exemplary principle for an IgG depletion assay;

FIG. 4 depicts a characterization of the dynamic range of a GNP binding assay;

FIG. 5 depicts levels anti-AraH2 IgE binding at varying levels of anti-AraH2 IgG4;

FIG. 6 depicts sIgE detection levels in control samples and samples that underwent IgG depletion using PG;

FIG. 7 depicts sIgE detection levels in control samples and samples that underwent IgG depletion using bPG and SA;

FIG. 8 depicts sIgE detection levels in control samples and samples that underwent IgG depletion using varying amounts of bPG and SA;

FIG. 9 depicts sIgE detection levels in multiple subject samples that were either treated or that underwent IgG depletion using bPG and SA;

FIG. 10 is a diagram depicting an exemplary method of allergen testing.

FIG. 11 is a diagram depicting an exemplary method of IgG depletion.

FIG. 12 is an exemplary embodiment of an apparatus for performing microfluidic-based biochemical assay;

FIGS. 13A-C are exemplary embodiments of sensor device integrated to different microfluidic environments;

FIGS. 14A-B is an exemplary embodiments of possible data transfer from apparatus to external device;

FIGS. 15A-B are exemplary embodiments, of an active flow component;

FIGS. 16A-C are exemplary embodiments of plurality of microfluidic features that may be utilized for both lateral and longitudinal mixing;

FIG. 17 is an exemplary embodiment of other types of mixing fluids using flow component;

FIGS. 18A-B are exemplary embodiments of a single-step assay performed using the apparatus;

FIG. 19 is an exemplary embodiment of a two-step assay performed using the apparatus;

FIG. 20 is an exemplary embodiment of a three-step assay performed using the apparatus;

FIG. 21 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for allergen detection and IgG depletion. In some embodiments, an allergen specific IgE antibody (sIgE) test may be used to determine whether a subject has allergic sensitization to one or more allergens. sIgE tests may function by detecting the presence of sIgE in a subject sample. In non-limiting examples, a subject sample may include a blood sample, a serum sample, or a plasma sample. In non-limiting examples, sIgE tests may include skin prick tests, intradermal tests, and patch tests. sIgE testing suffers a drawback of potential inaccuracy due to a failure of sufficient numbers of sIgE to bind to an allergen due to competition from allergen specific IgG antibodies (sIgG) specific for the same allergens. sIgG may bind to allergens, preventing sIgE from binding due to, for example, binding to the same target site or steric blocking. Introduction of an IgG depletion step may reduce sIgG levels in a subject sample, allowing sIgE to bind to an allergen such that sIgE binding may be detected.

IgE is a class of antibodies important to type I immediate allergic responses. High levels of IgE antibodies may be present in allergic or atopic individuals compared to non-allergic or non-atopic individuals. IgE antibodies may bind to high-affinity IgE receptor (FcεRI) on mast cells and basophils, and may bind to CD23 on B cells, triggering an immune response.

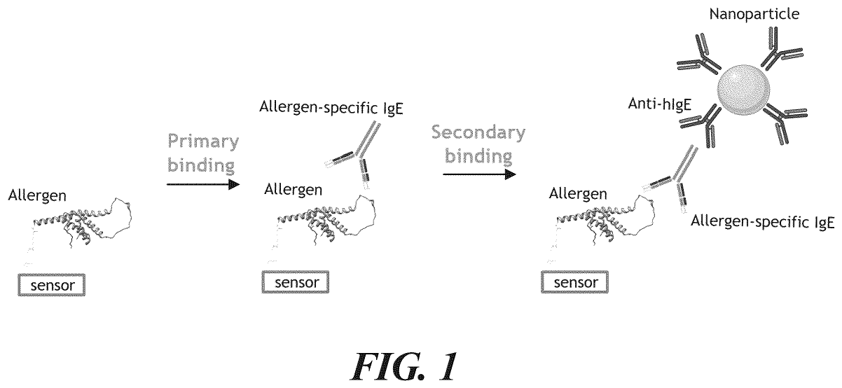

Now referring to FIG. 1, in some embodiments, sIgE levels may be detected using a sensor, including without limitation a chemical, electrochemical, and/or optical sensor. In some embodiments, sIgE levels may be detected using an optical sensor. In some embodiments, sIgE levels may be detected using a biolayer interferometry system. Biolayer interferometry is a technique for measuring molecular interactions by analyzing interference patterns of white light reflected from a sensor. Biolayer interferometry may function by immobilizing a first molecule, such as an allergen, to a sensor and exposing the sensor to a second molecule, such as a sample including sIgE. If the second molecule binds to the first molecule, this may be measured based on a shift in an interference pattern.

Still referring to FIG. 1, an allergen such as AraH2 may be immobilized on a sensor, and the sensor may be exposed to a sample including sIgE, such as sIgE specific for AraH2. This may lead to primary binding (of the sIgE to the allergen). A nanoparticle including an anti-hIgE binding site may be present in the sample or introduced into the sample. In some embodiments, a nanoparticle including an anti-hIgE binding site may include a nanoparticle whose presence or absence may be detected by a sensor, such as a biolayer interferometry system. In some embodiments, a nanoparticle may include a nanoparticle whose presence or absence affects optical properties of a fluid solution and/or element containing such solution, such as without limitation a fluid solution containing an analyte. Such optical properties may include a refractive index of fluid, resonant frequency and/or wavelength of an element containing the fluid, and the like. In a non-limiting example, a nanoparticle may be detectable using a ring resonator in fluid contact with an immobilized allergen.

Still referring to FIG. 1, in some embodiments, a nanoparticle may include a magnetic nanoparticle. In some embodiments, a nanoparticle may include a gold nanoparticle (GNP), silver nanoparticle, silicon nanoparticle, and the like. In some embodiments, a nanoparticle may include a gold nanoparticle. In some embodiments, a nanoparticle may include a 40 nm GNP.

Still referring to FIG. 1, in some embodiments, an anti-hIgE binding site may include an anti-IgE antibody conjugated to a nanoparticle. The presence of a nanoparticle including an anti-hIgE binding site may lead to secondary binding (of the anti-hIgE antibody to the sIgE-allergen complex). In some embodiments, this indirect binding of a nanoparticle to the sensor may be detected by the sensor. In some embodiments, as nanoparticles bind to allergens through sIgE, the effective refractive index of the surrounding medium may be changed, resulting in a shift in the resonant spectrum. In some embodiments, this shift in resonant wavelength may be detected by a biolayer interferometry system. In some embodiments, a change in light intensity, such as a change in light intensity at a particular wavelength, may be detected by a biolayer interferometry system.

Still referring to FIG. 1, in some embodiments, a method of allergen detection may include functionalizing a sensor with an allergen, such as an AraH2 protein. In some embodiments, a method may then include incubating the sensor functionalized with allergen with a sample containing anti-allergen IgE antibodies, such as anti-AraH2 IgE antibodies. In some embodiments, a method may then include incubating the sensor with GNP conjugated to anti-IgE antibodies.

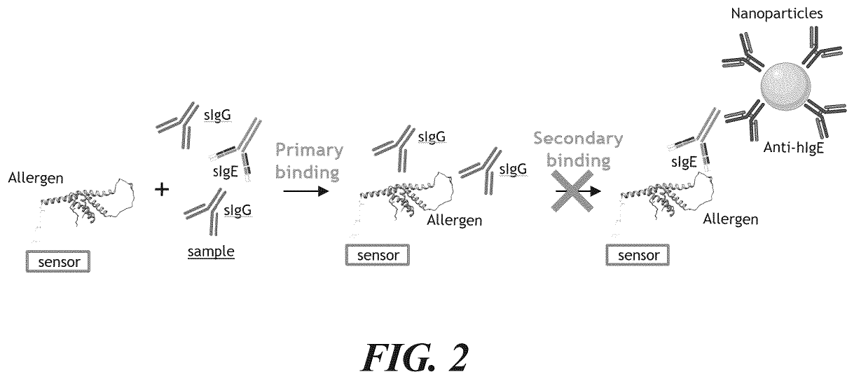

Now referring to FIG. 2, in some embodiments, a subject sample may contain sIgG. sIgG may bind to and/or block a sIgE binding site on an allergen by, for example, steric blocking. This competition may lead to a reduction in sIgE binding to allergen and, as a result, a reduction in indirect binding of nanoparticles to allergen. This may reduce the measured sensitivity of a subject sample to an allergen, potentially leading to a false negative result. This effect is especially relevant in those immunoassays where low levels of allergens are functionalized but gold standard sIgE immunoassays, where a high surface allergen density is achieved, also suffer.

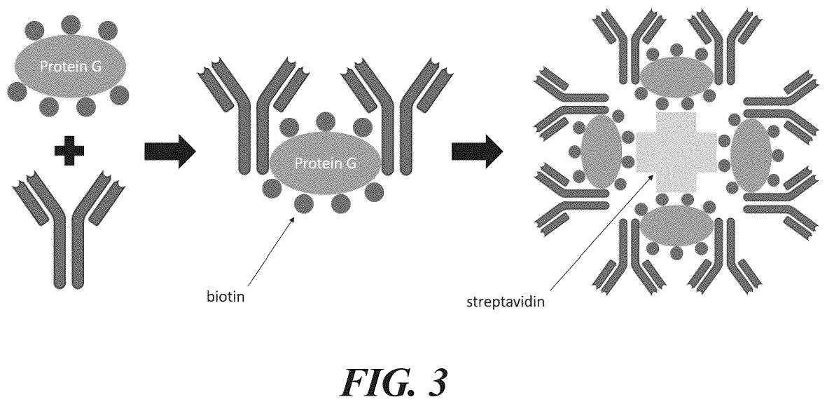

Now referring to FIG. 3, in some embodiments, incorporation of an IgG depletion step may reduce the competition by sIgG for sensor-bound allergens, allowing sIgE to bind. In some embodiments, an IgG depletion step may be performed on a subject sample. In non-limiting examples, an IgG depletion step may be done in a sample collection buffer or in a reagent pad.

Still referring to FIG. 3, a schematic representation of an exemplary IgG depletion assay is shown. In some embodiments, IgG depletion may include addition of an IgG binder. As used herein, an “IgG binder” is a molecule specific for IgG antibodies and not specific for IgE antibodies. In some embodiments, an IgG binder may include a plurality of binding sites for IgG antibodies. In some embodiments, an IgG binder may include 1, 2, 3, 4, 5, 6, or more IgG binding sites. In some embodiments, an IgG binder may include Protein A. In some embodiments, an IgG binder may include Protein G (PG). PG includes 2 binding sites for IgG Fc fragments, allowing it to bind 2 IgG together. In some embodiments, an IgG binder may include Biotinylated Protein G (bPG), which also contains 2 IgG Fc binding sites.

Still referring to FIG. 3, in some embodiments, IgG depletion may include addition of an IgG binder aggregator. As used herein, an “IgG binder aggregator” is a molecule that includes a plurality of binding sites specific for an IgG binder. In some embodiments, an IgG binder aggregator may include a plurality of binding sites for IgG binders. In some embodiments, an IgG binder aggregator may include 1, 2, 3, 4, 5, 6, or more binding sites specific for IgG binders. In some embodiments, an IgG binder aggregator may bind to a different location on an IgG binder than the IgG binding site. In some embodiments, binding of an IgG binder aggregator to an IgG binder may not block binding of IgG binder to an IgG antibody. In some embodiments, an IgG aggregator may include streptavidin (SA). SA includes 4 binding sites for biotin. In some embodiments, bPG may bind to SA, and one or more IgG, creating a bPG/IgG/SA complex. In some embodiments, a bPG/IgG/SA may have a size of ˜1,340 kDa, which may be filtered out using a size exclusion process. In some embodiments, a size exclusion process may include centrifugation, such as quick spin centrifugation. In some embodiments, a size exclusion process may include a 10 nm (or lower) filter. In some embodiments, a filter may include a 10 nm filter, a 5 nm filter, a 1 nm filter, or a smaller filter.

Still referring to FIG. 3, in some embodiments, IgG may be precipitated from a subject sample using a specific biotinylated immunoglobulin-binding protein, such as Protein A or Protein G in combination with streptavidin and may be pulled down and/or removed using, in non-limiting examples, tabletop centrifuge or filtration. In some embodiments, IgG may be precipitated from a subject sample using a specific non-biotinylated immunoglobulin-binding protein, such as Protein A or Protein G and may be pulled down and/or removed using, in non-limiting examples, tabletop centrifuge or filtration. In some embodiments, IgG may be precipitated from a subject sample using a specific non-biotinylated immunoglobulin-binding protein, such as such as Protein A or Protein G, using an optimum tIgG:PG molar ratio of 2.0; cross-linked complexes may then be pulled down and/or removed using tabletop centrifuge or filtration. In some embodiments, IgG may be precipitated from a subject sample using specific biotinylated immunoglobulin-binding protein, such as Protein A or Protein G, in combination with streptavidin, using an optimum tIgG:PG molar ratio of 2.0, and an optimum PG:streptavidin molar ratio of 2.0; cross-linked complexes may then be pulled down and/or removed using tabletop centrifuge or filtration.

Still referring to FIG. 3, in some embodiments, Protein G may be selected as an IgG binder over Protein A. Protein G does not bind to IgE antibodies, but Protein A does. In order to avoid removing IgE antibodies from a sample, in some embodiments, Protein G may be used as an IgG binder. In some embodiments, low tIgG:PG molar ratios (<2.0) promotes aggregation of IgE and by extension sIgE molecules while high tIgG:PG molar ratios (>2.0) produces insufficient IgG aggregation, and thus remaining sIgG may be high.

Still referring to FIG. 3, in surface-based assays such as those on ring resonator sensors or BLI sensors like the Gator-Bio, diffusion rates are important when there is not enough time to reach equilibrium, thus the size of the binding molecule has an effect on the kinetics. By creating aggregates of IgG molecules, we effectively slow down their binding kinetics and thus make aggregates that are not filtered or precipitated out less likely to bind to the sensor than the smaller unaggregated IgEs. Thus, signal recovery may be observed even without filtration of the sample after aggregation. In some embodiments, a method step may not include a step in which compounds are removed from a sample as a function of their size. In some embodiments, omission of such a step may allow for a simpler process and/or testing kit.

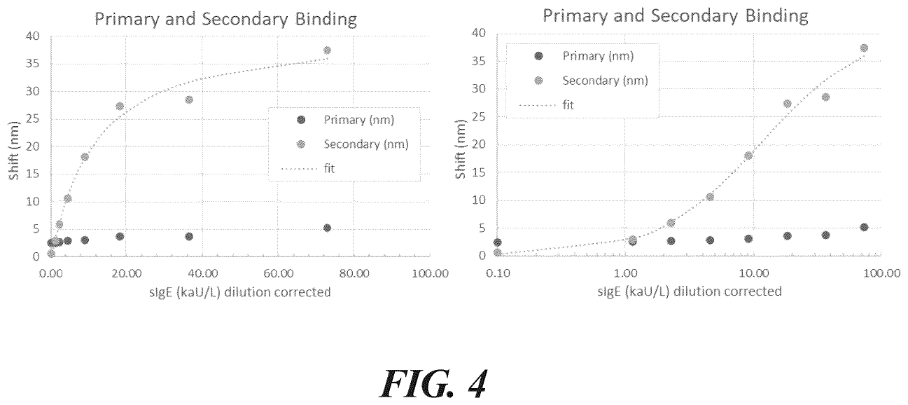

Now referring to FIG. 4, the dynamic range of an assay was characterized, and the results are presented in FIG. 4. AR (Amino Reactive) probes, also called sensors, functionalized with AraH2 protein were incubated with 50% of a subject serum sample with known sIgE of 72.9 kU/L in 3.5C buffer which was further serially diluted in an Ig-free serum. Sensors were transferred into a 200 pM GNP solution, and GNP binding was analyzed using GatorBio. 1-100 kU/L of anti-AraH2 IgE was detected using this assay set up.

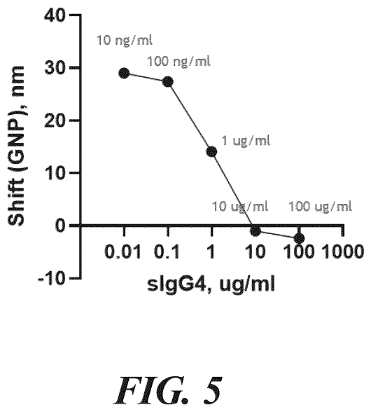

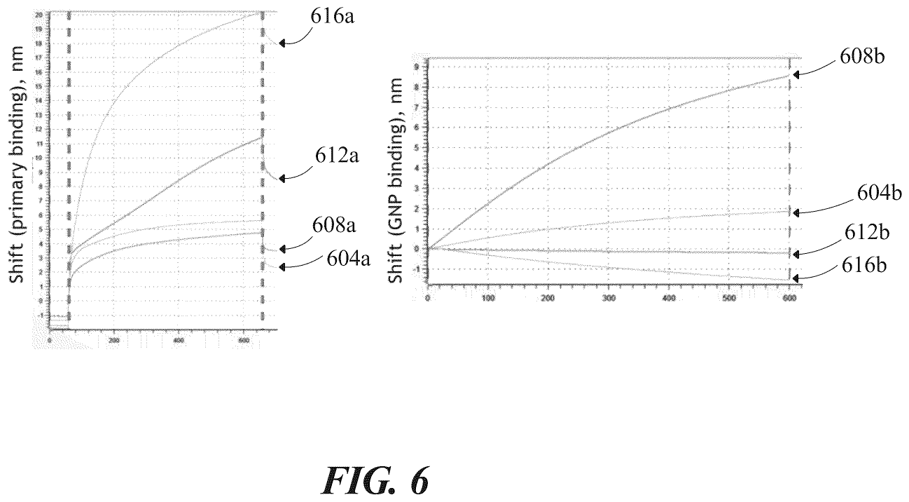

Now referring to FIG. 5, sIgG interference on sIgE quantification was assessed, and the results are presented in FIG. 5. Sensors functionalized with AraH2 protein were incubated in PBS/EDTA/Tween-20 buffer spiked with 100 kU/L of monoclonal human anti-AraH2 IgE and 0-100 ug/ml of monoclonal human anti-AraH2 IgG4 targeting the same epitope as the IgE. Next, sensors were transferred to 200 pM GNP and GNP binding to the sensor surface was measured using GatorBio. As shown in FIG. 5, IgG4 concentrations higher than 100 ng/ml resulted in noticeable decrease in GNP binding to the sensor, perturbing quantification of anti-AraH2 IgE.

Still referring to FIG. 5, in some embodiments, both sIgG and sIgG4 concentrations can be higher than sIgE concentrations. In some embodiments, ratios at the highest concentration are ˜1,250 for sIgG/sIgE, and ˜150 for sIgG4/sIgE. Thus, for clinically meaningful sIgE levels of 0.35-100 kU/L (0.8-244 ng/ml), sIgG concentration can be 0.882-256.2 ug/ml, which is much higher than the concentration at which sIgG interfere in our assay (100 ng/ml).

Now referring to FIG. 6, we tested whether IgG depletion using PG can recover sIgE detection in subject samples. Two subject serum samples containing 36.4 kU/L of sIgE and 12.2 ug/ml of sIgG (subject 1, indicated by 612 and 604 on FIG. 6) or 55.4 kU/L of sIgE and 63.1 ug/ml of sIgG (subject 2, indicated by 616 and 608 on FIG. 6) were divided in two equal parts. One half was purified using PG spin column (indicated by 604 and 608 on FIG. 6), other half was used as a control (indicated by 612 and 616 on FIG. 6). Sensors functionalized with AraH2 protein were incubated with controls or purified samples, washed in PBS to decrease the level of non-specific binding from the serum, and then transferred to 200 pM GNP. GNP binding to the sensor surface measured using GatorBio. Average reported amount of IgG in human serum is ˜15 mg/ml. As shown by FIG. 6, primary binding to AraH2 sensor was noticeably decreased in PG purified samples. Which was probably due to decreased amount of IgG in the serum. GNP binding was increased in PG treated samples presumably because of increased sIgE binding compared to unpurified control.

Still referring to FIG. 6, despite encouraging results, spin columns are long and cumbersome option for point-of-care diagnostics, as it requires multiple steps and availability of at least a benchtop centrifuge. In some embodiments, a single tube purification method such as bPG-SA method described below may be used over centrifugation. In some embodiments, a bPG-SA method may be more suitable for point-of-care or even at-home diagnostics.

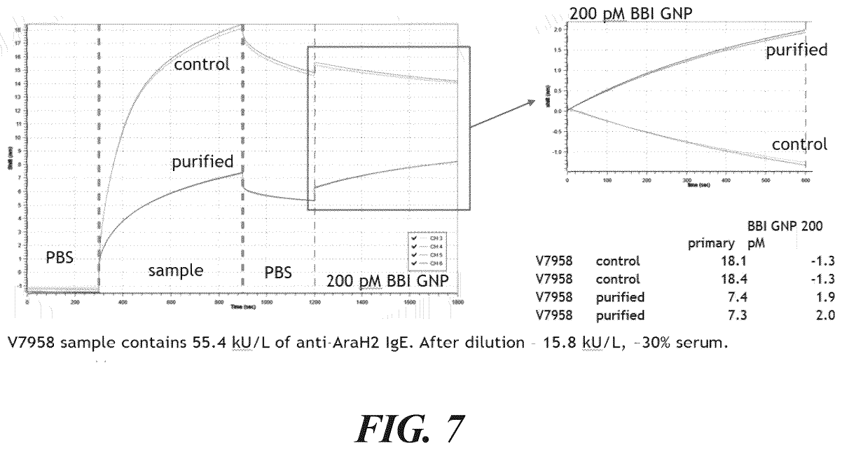

Now referring to FIG. 7, bPG-SA method was tested on subject samples. Subject serum sample containing 55.4 kU/L of sIgE and 63.1 of sIgG was divided in two equal parts. One half was purified using bPG-SA method, and the other half was used as a control. Sensors functionalized with AraH2 protein were incubated with the controls or purified sample, washed in PBS to decrease the level of non-specific binding from the serum, and then transferred to 200 pM GNP. GNP binding to the sensor surface measured using GatorBio. As shown by FIG. 7, purified sample demonstrated evidently lower level of primary binding to AraH2 sensor and recovered signal from anti-AraH2 IgE binding.

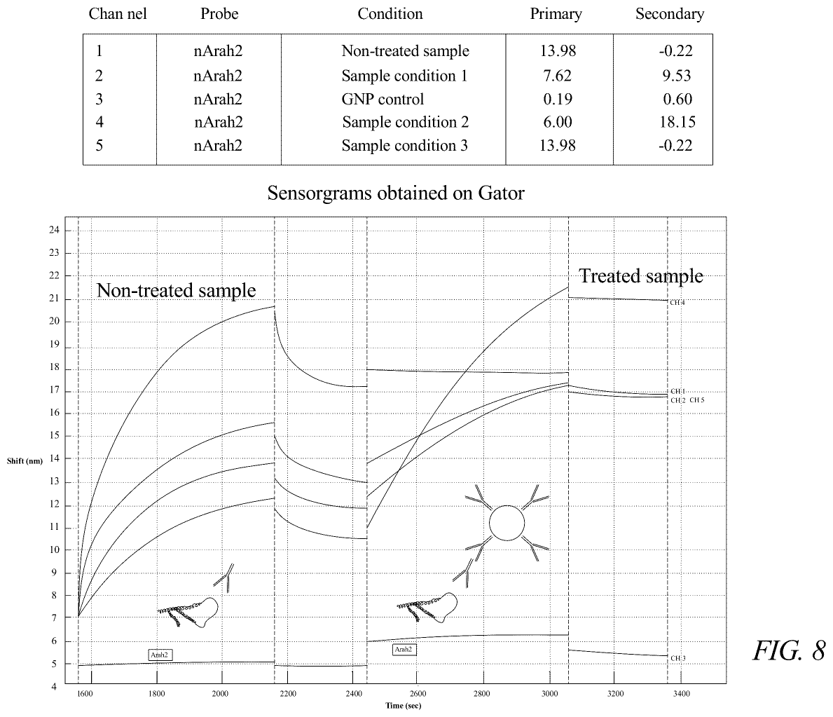

Now referring to FIG. 8, bPG-SA method was tested using varying molar ratios. The same subject serum containing 55.4 kU/L of sIgE and 63.1 μg/mL of sIgG was used. Sensors functionalized with AraH2 protein were incubated with the control and three different bPG-SA conditions, washed in PBS to decrease the level of non-specific binding from the serum, and then transferred to 200 pM GNP. GNP binding to the sensor surface measured using GatorBio. As shown in FIG. 8, the non-treated sample has no secondary shift. In contrast, condition 2, which had a tIgG:PG molar ratio of 2.0 produced a secondary shift of 18.15 nm.

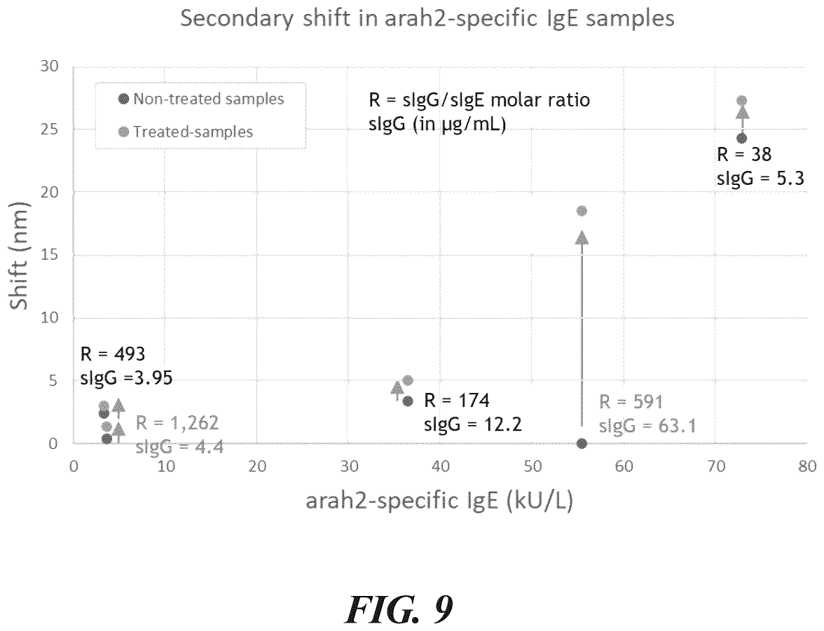

Now referring to FIG. 9, bPG-SA strategy was tested using multiple allergic subject samples. Samples were plotted according to their sIgE content. In each of the samples the sIgG/sIgE molar ratio is shown together with the sIgG amount in μg/mL. The positive impact on the sIgE signal of different patient samples is shown in FIG. 9. From this figure it is clear that completely inhibited samples (undetectable initial sIgE content on GatorBio) can be recuperated and that the amount of recovered sIgE signal is dependent on the sIgG/sIgE molar ratio and the total sIgG content of the sample.

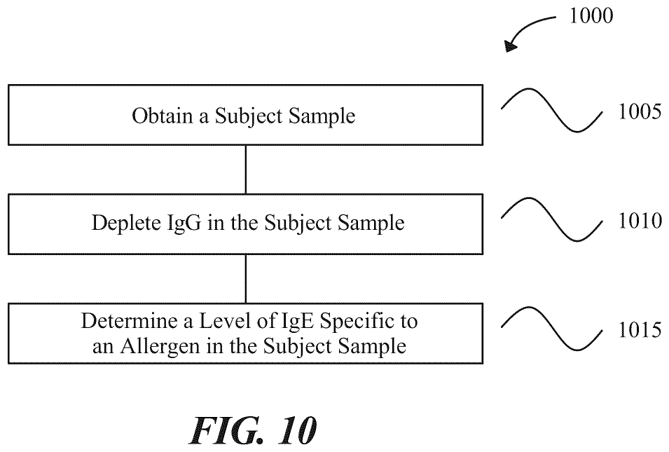

Now referring to FIG. 10, an exemplary embodiment of method 1000 is illustrated. In some embodiments, method 1000 may include obtaining a subject sample 1005.

Still referring to FIG. 10, in some embodiments, method 1000 may include depleting IgG in a subject sample 1010. In some embodiments, depleting IgG in a subject sample 1010 may include adding an IgG binder to the subject sample; and adding an IgG binder aggregator to the subject sample. In some embodiments, an IgG binder is bPG. In some embodiments, an IgG binder aggregator is SA. In some embodiments, bPG is added at a tIgG:PG molar ratio of 2.0 and SA is added at a PG: SA molar ratio of 2.0.

Still referring to FIG. 10, in some embodiments, method 1000 may include determining a level of IgE specific to an allergen in a subject sample 1015. In some embodiments, determining a level of IgE specific to an allergen in a subject sample is done using a biolayer interferometry system. In some embodiments, a biolayer interferometry system is configured to detect binding of a gold nanoparticle to a sensor. In some embodiments, a gold nanoparticle is a 40 nm gold nanoparticle.

Still referring to FIG. 10, in some embodiments, method 1000 may further include removing compounds from a subject sample as a function of their size. In some embodiments, removing compounds from the subject sample as a function of their size includes quick spin centrifugation. In some embodiments, removing compounds from the subject sample as a function of their size includes filtration using a 10 nm filter.

Still referring to FIG. 10, in some embodiments, a subject sample includes subject blood, subject serum, or subject plasma. In some embodiments, an allergen may include an allergen from a source selected from the list consisting of alder (Alnus incana), birch (Betula alba/verrucosa), cypress (Cupressus sempervirens/arizonica), hazel (Corylus avellana), plane (Platanus vulgaris), grass mix (Poa pratensis, Dactilis glomerata, Lolium perenne, Phleum pratense, Festuca pratensis, Helictotrichon pratense), olive (Olea europea), mugwort (Artemisia vulgaris), ragweed (Ambrosia artemisiifolia), Alternaria alternata (tenuis), Cladosporium herbarum, Aspergillus fumigatus, Parietaria, cat (Felis domesticus), dog (Canis familiaris), dust mite (Dermatophagoides pteronyssinus/farinae), cockroach (Blatella germanica), milk, eggs, fish, crustacean shellfish, tree nuts, peanuts, wheat, and soybeans.

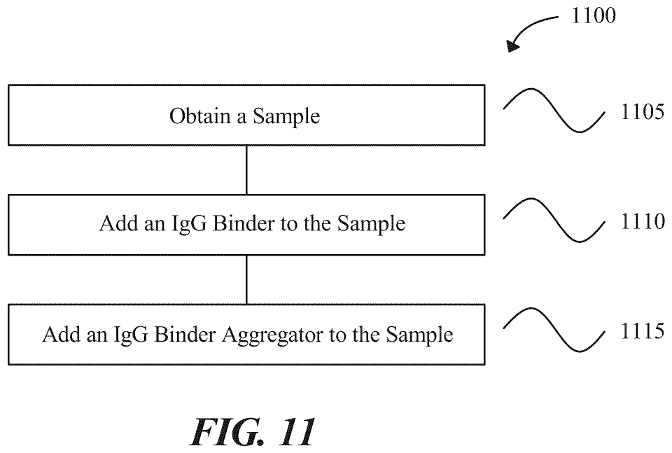

Now referring to FIG. 11, an exemplary embodiment of method 1100 is illustrated. In some embodiments, method 1100 may include obtaining a sample 1105.

Still referring to FIG. 11, in some embodiments, method 1110 may include adding an IgG binder to the sample. In some embodiments, an IgG binder is bPG.

Still referring to FIG. 11, in some embodiments, method 1115 may include adding an IgG binder aggregator to the sample. In some embodiments, an IgG binder aggregator is SA. In some embodiments, bPG is added at a tIgG:PG molar ratio of 2.0 and SA is added at a PG:SA molar ratio of 2.0.

Still referring to FIG. 11, in some embodiments, method 1115 may further include removing compounds from a sample as a function of their size. In some embodiments, removing compounds from the subject sample as a function of their size includes quick spin centrifugation. In some embodiments, removing compounds from the subject sample as a function of their size includes filtration using a 10 nm filter.

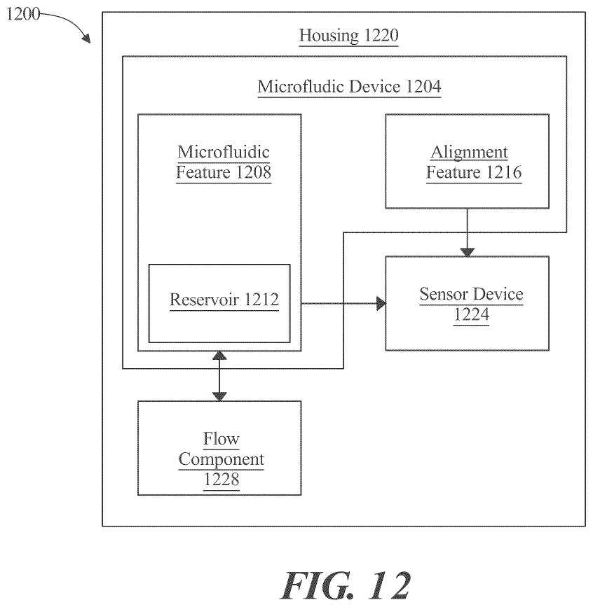

Now referring to FIG. 12, an exemplary embodiment of apparatus 1200 is illustrated. In some embodiments, apparatus 1200 may be used to detect binding of a nanoparticle to a sensor through sIgE. For example, apparatus 1200 may be used to detect binding of a nanoparticle conjugated to an anti-IgE antibody to a sIgE antibody, which is bound to an allergen affixed to a sensor.

Still referring to FIG. 12, an exemplary embodiment of an apparatus 1200 for performing microfluidic-based biochemical assays is illustrated. As used in this disclosure, a “microfluidic-based biochemical assay” is an assay on small volumes (i.e., in unit of ml or nl) of fluids. In some embodiments, microfluidic-based biochemical assay may be used for a wide range of applications, such as without limitation, medical diagnostics, drug discovery, environmental monitoring, and food safety testing, and the like. Apparatus 1200 may include a computing device. Computing device may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device may include a single computing device operating independently, or may include two or more computing device operating in concert, in parallel, sequentially or the like; two or more computing devices may be included together in a single computing device or in two or more computing devices. Computing device may interface or communicate with one or more additional devices as described below in further detail via a network interface device. Network interface device may be utilized for connecting computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device may include but is not limited to, for example, a computing device or cluster of computing devices in a first location and a second computing device or cluster of computing devices in a second location. Computing device may include one or more computing devices dedicated to data storage, security, distribution of traffic for load balancing, and the like. Computing device may distribute one or more computing tasks as described below across a plurality of computing devices of computing device, which may operate in parallel, in series, redundantly, or in any other manner used for distribution of tasks or memory between computing devices. Computing device may be implemented using a “shared nothing” architecture in which data is cached at the worker, in an embodiment, this may enable scalability of apparatus 1200 and/or computing device.

With continued reference to FIG. 12, computing device may be designed and/or configured to perform any method, method step, or sequence of method steps in any embodiment described in this disclosure, in any order and with any degree of repetition. For instance, computing device may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 12, apparatus 1200 includes a microfluidic device 1204. As used in this disclosure, a “microfluidic device” is a device that is configured to act upon fluids at a small scale, such as without limitation a sub-millimeter scale. At small scales, surface forces may dominate volumetric forces. In a non-limiting example, microfluidic device may be consistent with any microfluidic device described in U.S. patent application Ser. No. 17/859,932, filed on Jul. 7, 2022, entitled “SYSTEM AND METHODS FOR FLUID SENSING USING PASSIVE FLOW,” the entirety of which is incorporated herein by reference.

With continued reference to FIG. 12, microfluidic device 1204 includes at least a microfluidic feature 1208. As used in this disclosure, a “microfluidic feature” is a structure within microfluidic device 1204 that is designed and/or configured to manipulate one or more fluids at micro scale. In a non-limiting example, microfluidic feature 1208 may include, without limitation, reservoir, microfluidic channel, conjugate pad, and the like as described in further detail below in this disclosure. In some cases, microfluidic feature 1208 may enable a precise manipulation of fluids and samples in a controlled and/or reproducible manner within microfluidic device 1204. In some embodiments, microfluidic feature 1208 of microfluidic device 1204 may be designed and arranged based on particular needs of a given microfluidic-based biochemical assay. In other embodiments, microfluidic feature 1208 of microfluidic device 1204 may be varied depending on the type of the at least a fluid being used, that is directly contact with microfluidic feature 1208. In a non-limiting example, attributes of microfluidic feature 1208 such as, without the size and/or shape of the substrate may be determined as a function of specific assay protocols. Exemplary embodiments of microfluidic feature 1208 are described in further detail below in this disclosure.

With continued reference to FIG. 12, microfluidic feature 1208 includes at least a reservoir 1212. Reservoir 1212 may be configured to contain at least a fluid. In a non-limiting example, fluid may include a sample fluid to be analyzed from a subject; for instance, and without limitation, reservoir 1212 of microfluidic device 1204 may contain a blood sample taken from a patient. Alternatively, or additionally, fluid may include one or more suspensions and/or solutions of reagents, molecules, or other items to be analyzed and/or utilized, including without limitation monomers such as individual nucleotides, amino acids, or the like, one or more buffer solutions and/or saline solutions for rinsing steps, and/or one or more analytes to be detected and/or analyzed. Fluid and/or microfluidic device may be used, without limitation, in processes as disclosed in U.S. Nonprovisional application Ser. No. 18/107,135, filed on Feb. 8, 2023, and titled “APPARATUS AND METHODS FOR ACTUATING FLUIDS IN A BIOSENSOR CARTRIDGE,” which is incorporated by reference in its entirety. Reservoir 1212 may have at least an inlet, at least an outlet, or both. Reservoir 1212 may further include, without limitation, a well, a channel, a flow path, a flow cell, a pump, and the like. In a non-limiting example, fluid may be input through the at least an inlet into reservoir 1212 and/or output through the at least an outlet. At least an outlet may be connected to other components and/or devices within microfluidic device 1204; for instance, and without limitation, at least an outlet may be connected to other microfluidic feature 1208 such as microfluidic channel as described below in this disclosure.

With continued reference to FIG. 12, microfluidic device 1204 includes at least an alignment feature 1216 at a distance from at least a microfluidic feature 1208. In a microfluidic device used for performing biochemical assays, an “alignment feature” is a physical feature that helps to precisely align components of microfluidic device 1204 with other components. Alignment feature 1216 is configured for precise positioning and attaching a sensor device, wherein the sensor device may include any sensor device as described in this disclosure. In some embodiments, alignment feature 1216 may be configured for precise positioning and attaching other components external to apparatus 1200; for instance, and without limitation, without limitation, an external device may be coupled with apparatus 1200 through one or more alignment features 1216, such as, without limitation, a multi-fiber push connector (MPO), bracket, press fastener (with spring mechanism) and the like as described in further detail below. In some embodiments, alignment feature 1216 may be configured for precise positioning microfluidic feature 1208; for instance, and without limitation, microfluidic channel may be etched along alignment feature 1216 during etching process as described below. In some cases, microfluidic channel may be configured to be in parallel to alignment feature 1216 at a distance. In other cases, microfluidic channel may be configured to be perpendicular to alignment feature 1216 at a distance. Other embodiments of microfluidic feature 1208 alignment employing alignment feature 1216 as reference may include, without limitation, symmetrical alignment, relative positioning, fix positioning, and the like thereof.

With continued reference to FIG. 12, in some embodiments, alignment feature 1216 may include a housing 1200. As used in this disclosure, a “housing” refers to an outer structure configured to contain a plurality of components, such as, without limitation, components of apparatus 1200 as described in this disclosure. In a non-limiting example, alignment feature 1216 may include an outer casing of apparatus 1200. In some cases, housing 1220 may be made from a durable, lightweight material such as without limitation, plastic, metal, and/or the like. In some embodiments, housing 1220 may be designed and configured to protect sensitive components of apparatus 1200 from damage or contamination. In a non-limiting example, at least an alignment feature 1216 may include one or more flat facets located on housing 1220 configured to constraint at least a sensor device as described above in this disclosure, wherein the “flat facet” refers to a surface or object that is smooth and event, without any significant curvature or bumps. In another non-limiting example, at least an alignment feature 1216 may include one or more physical notches and/or grooves that allow for precise placement of devices and/or components. In yet another non-limiting example, at least an alignment feature 1216 may include one or more optical markers or alignment indicators that are visible (through human eye, microscope, any other imaging system, and/or the like) and allow for accurate positioning of devices and/or components. In a further non-limiting examples, at least an alignment feature 1216 may include one or more tapered or angled surfaces (of housing 1220) that guide the one or more microfluidic features 1208 through apparatus 1200. In other non-limiting example, housing 1220 may include one or more surface coatings and/or modifications that reduce the likelihood of unwanted adhesion or interference with external components such as, without limitation, external device as described in further detail below. Additionally, or alternatively, at least an alignment feature 1216 may further include features such as latches, clips, or other fasteners that help to secure apparatus 1200 in place during use.

Still referring to FIG. 12, in some embodiments, at least an alignment feature 1216 may include a sealer. As used in this disclosure, a “sealer” is a component that is used to create a secure seal between components of apparatus 1200. In some cases, sealer may be configured to prevent contamination (i.e., dust, debris, other external factors, and/or the like) of the fluids, thus ensuring accurate, reliable results. In a non-limiting example, sealer of at least an alignment feature 1216 may be configured to seal between one or more microfluidic features 1208 within microfluidic device 1204 and housing 1220. In some embodiments, sealers can take many forms, depending on the overall design and/or configuration of apparatus 1200; for instance, and without limitation, sealer may include O-rings, gaskets, adhesives, or other materials that are used to fill gaps and/or create a fluid-tight seal between microfluidic channel and housing 1220. In some embodiments, sealer may be applied to the surface of microfluidic device 1204 to create a barrier between microfluidic feature 1208 with external environment. In some cases, sealer may be heat-sealable. In a non-limiting example, sealer may include a heat-sealable film or tape, made from a flexible, thermoplastic material that can be heated and molded to the contours of the apparatus 1200, creating a barrier between the microfluidic device 1204 and the external environment.

With continued reference to FIG. 12, apparatus 1200 includes a sensor device 1224. Sensor device 1224 may be configured to be in sensed communication with at least a fluid contained within or otherwise acted upon by microfluidic feature 1208. As used in this disclosure, a “sensor device” is one or more independent sensors, as described herein, where any number of the described sensors may be used to detect any number of physical quantities associated with an microfluidic environment. In some embodiments, sensor device 1224 may include an optical device. As used in this disclosure, an “optical device” is any device that generates, transmits, detects, or otherwise functions using electromagnetic radiation, including without limitation ultra-violet light, visible light, near infrared light, infrared light, and the like. In some embodiments, optical device may include one or more waveguide. As used in this disclosure, a “waveguide” is a component that is configured to propagate electromagnetic radiation, including without limitation ultra-violet light, visible light, near infrared light, infrared light, and the like. A waveguide may include a lightguide, a fiberoptic, or the like. A waveguide may include a grating within a transmissive material. In some cases, a waveguide may be configured to function as one or more optical devices, for example a resonator (e.g., microring resonator), an interferometer, or the like. In some cases, waveguide may be configured to propagate an electromagnetic radiation (EMR). In a non-limiting example, sensor device 1224 may include any sensor device described in U.S. patent application Ser. No. 17/859,932 and/or any other disclosure incorporate by reference herein. Sensor device 1224 may include a sensor, wherein the sensor may be optical communication with one or more waveguide. Such sensor may be configured to detect a variance in at least an optical property associated with the at least a fluid. As used in this disclosure, an “optical property” is any detectable characteristic associated with electromagnetic radiation, for instance UV, visible light, infrared, and the like. In some cases, sensor device may generate and/or communicate signal representative of the detected property.

Still referring to FIG. 12, in some embodiments, sensor may be in communication with the computing device. For instance, and without limitation, sensor 1224 may communicate with computing device using one or more signals. As used in this disclosure, a “signal” is a human-intelligible and/or machine-readable representation of data, for example and without limitation an electrical and/or digital signal from one device to another; signals may be passed using any suitable communicative connection. As used in this disclosure, “communicatively connected” means connected by way of a connection, attachment, or linkage between two or more relata which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure. A signal may include an optical signal, a hydraulic signal, a pneumatic signal, a mechanical signal, an electric signal, a digital signal, an analog signal and the like. In some cases, a signal may be used to communicate with a computing device, for example by way of one or more ports. In some cases, a signal may be transmitted and/or received by computing device, for example by way of an input/output port. An analog signal may be digitized, for example by way of an analog to digital converter. In some cases, an analog signal may be processed, for example by way of any analog signal processing steps described in this disclosure, prior to digitization. In some cases, a digital signal may be used to communicate between two or more devices, including without limitation computing devices. In some cases, a digital signal may be communicated by way of one or more communication protocols, including without limitation internet protocol (IP), controller area network (CAN) protocols, serial communication protocols (e.g., universal asynchronous receiver-transmitter [UART]), parallel communication protocols (e.g., IEEE 128 [printer port]), and the like.

Still referring to FIG. 12, in some cases, apparatus 1200, sensor, and/or computing device may perform one or more signal processing steps on a signal. For instance, apparatus 1200, sensor, and/or computing device may analyze, modify, and/or synthesize a signal representative of data in order to improve the signal, for instance by improving transmission, storage efficiency, or signal to noise ratio. Exemplary methods of signal processing may include analog, continuous time, discrete, digital, nonlinear, and statistical. Analog signal processing may be performed on non-digitized or analog signals. Exemplary analog processes may include passive filters, active filters, additive mixers, integrators, delay lines, compandors, multipliers, voltage-controlled filters, voltage- controlled oscillators, phase-locked loops, and/or any other process using operational amplifiers or other analog circuit elements. Continuous-time signal processing may be used, in some cases, to process signals which vary continuously within a domain, for instance time. Exemplary non-limiting continuous time processes may include time domain processing, frequency domain processing (Fourier transform), and complex frequency domain processing. Discrete time signal processing may be used when a signal is sampled non-continuously or at discrete time intervals (i.e., quantized in time). Analog discrete-time signal processing may process a signal using the following exemplary circuits sample and hold circuits, analog time-division multiplexers, analog delay lines and analog feedback shift registers. Digital signal processing may be used to process digitized discrete-time sampled signals. Commonly, digital signal processing may be performed by a computing device or other specialized digital circuits, such as without limitation an application specific integrated circuit (ASIC), a field-programmable gate array (FPGA), or a specialized digital signal processor (DSP). Digital signal processing may be used to perform any combination of typical arithmetical operations, including fixed-point and floating-point, real-valued and complex-valued, multiplication and addition. Digital signal processing may additionally operate circular buffers and lookup tables. Further non-limiting examples of algorithms that may be performed according to digital signal processing techniques include fast Fourier transform (FFT), finite impulse response (FIR) filter, infinite impulse response (IIR) filter, and adaptive filters such as the Wiener and Kalman filters. Statistical signal processing may be used to process a signal as a random function (i.e., a stochastic process), utilizing statistical properties. For instance, in some embodiments, a signal may be modeled with a probability distribution indicating noise, which then may be used to reduce noise in a processed signal.

With continued reference to FIG. 12, in some embodiments, apparatus 1200 may include one or more light sources. As used in this disclosure, a “light source” is any device configured to emit electromagnetic radiation, such as without limitation light, UV, visible light, and/or infrared light. In some cases, a light source may include a coherent light source, which is configured to emit coherent light, for example a laser. In some cases, a light source may include a non-coherent light source configured to emit non-coherent light, for example a light emitting diode (LED). In some cases, light source may emit a light having substantially one wavelength. In some cases, light source may emit a light having a wavelength range. Light may have a wavelength in an ultraviolet range, a visible range, a near-infrared range, a mid-infrared range, and/or a far-infrared range. For example, in some cases light may have a wavelength within a range from about 100 nm to about 20 micrometers. In some cases, light may have a wavelength within a range of about 400 nm to about 2,500 nm. Light sources may include, one or more diode lasers, which may be fabricated, without limitation, as an element of an integrated circuit; diode lasers may include, without limitation, a Fabry Perot cavity laser, which may have multiple modes permitting outputting light of multiple wavelengths, a quantum dot and/or quantum well-based Fabry Perot cavity laser, an external cavity laser, a mode-locked laser such as a gain-absorber system, configured to output light of multiple wavelengths, a distributed feedback (DFB) laser, a distributed Bragg reflector (DBR) laser, an optical frequency comb, and/or a vertical cavity surface emitting laser. Light source may additionally or alternatively include a light-emitting diode (LED), an organic LED (OLED) and/or any other light emitter. In some cases, light source may be configured to couple light into optical device, for instance into one or more waveguide described above.

With continued reference to FIG. 12, in some embodiments, at least a sensor device 1224 may include at least a photodetector. In some cases, at least a sensor device 1224 may include a plurality of photodetectors, for instance at least a first photodetector and at least a second photodetector. In some cases, at least a first photodetector and/or at least a second photodetector may be configured to measure one or more of first optical output and second optical output, from a first waveguide and a second waveguide, respectively. As used in this disclosure, a “photodetector” is any device that is sensitive to light and thereby able to detect light. In some cases, a photodetector may include a photodiode, a photoresistor, a photosensor, a photovoltaic chip, and the like. In some cases, photodetector may include a Germanium-based photodiode. Light detectors may include, without limitation, Avalanche Photodiodes (APDs), Single Photon Avalanche Diodes (SPADs), Silicon Photomultipliers (SiPMs), Photo-Multiplier Tubes (PMTs), Micro-Channel Plates (MCPs), Micro-Channel Plate Photomultiplier Tubes (MCP-PMTs), Indium gallium arsenide semiconductors (InGaAs), photodiodes, and/or photosensitive or photon-detecting circuit elements, semiconductors and/or transducers. Avalanche Photo Diodes (APDs), as used herein, are diodes (e.g., without limitation p-n, p-i-n, and others) reverse biased such that a single photon generated carrier can trigger a short, temporary “avalanche” of photocurrent on the order of milliamps or more caused by electrons being accelerated through a high field region of the diode and impact ionizing covalent bonds in the bulk material, these in turn triggering greater impact ionization of electron-hole pairs. APDs provide a built-in stage of gain through avalanche multiplication. When the reverse bias is less than the breakdown voltage, the gain of the APD is approximately linear. For silicon APDs this gain is on the order of 10-100. Material of APD may contribute to gains. Germanium APDs may detect infrared out to a wavelength of 1.7 micrometers. InGaAs may detect infrared out to a wavelength of 1.6 micrometers. Mercury Cadmium Telluride (HgCdTe) may detect infrared out to a wavelength of 14 micrometers. An APD reverse biased significantly above the breakdown voltage is referred to as a Single Photon Avalanche Diode, or SPAD. In this case the n-p electric field is sufficiently high to sustain an avalanche of current with a single photon, hence referred to as “Geiger mode.” This avalanche current rises rapidly (sub-nanosecond), such that detection of the avalanche current can be used to approximate the arrival time of the incident photon. The SPAD may be pulled below breakdown voltage once triggered in order to reset or quench the avalanche current before another photon may be detected, as while the avalanche current is active carriers from additional photons may have a negligible effect on the current in the diode. At least a first photodetector may be configured to generate a first signal as a function of variance of an optical property of the first waveguide, where the first signal may include without limitation any voltage and/or current waveform. Additionally, or alternatively, sensor device may include a second photodetector located down beam from a second waveguide. In some embodiments, second photodetector may be configured to measure a variance of an optical property of second waveguide and generate a second signal as a function of the variance of the optical property of the second waveguide.

With continued reference to FIG. 12, in some cases, photodetector may include a photosensor array, for example without limitation a one-dimensional array. Photosensor array may be configured to detect a variance in an optical property of waveguide. In some cases, first photodetector and/or second photodetector may be wavelength dependent. For instance, and without limitation, first photodetector and/or second photodetector may have a narrow range of wavelengths to which each of first photodetector and second photodetector are sensitive. As a further non-limiting example, each of first photodetector and second photodetector may be preceded by wavelength-specific optical filters such as bandpass filters and/or filter sets, or the like; in any case, a splitter may divide output from optical matrix multiplier as described below and provide it to each of first photodetector and second photodetector. Alternatively, or additionally, one or more optical elements may divide output from waveguide prior to provision to each of first photodetector and second photodetector, such that each of first photodetector and second photodetector receives a distinct wavelength and/or set of wavelengths. For example, and without limitation, in some cases a wavelength demultiplexer may be disposed between waveguides and first photodetector and/or second photodetector; and the wavelength demultiplexer may be configured to separate one or more lights or light arrays dependent upon wavelength. As used in this disclosure, a “wavelength demultiplexer” is a device that is configured to separate two or more wavelengths of light from a shared optical path. In some cases, a wavelength demultiplexer may include at least a dichroic beam splitter. In some cases, a wavelength demultiplexer may include any of a hot mirror, a cold mirror, a short-pass filter, a long pass filter, a notch filter, and the like. An exemplary wavelength demultiplexer may include part No. WDM-11P from OZ Optics of Ottawa, Ontario, Canada. Further examples of demultiplexers may include, without limitation, gratings, prisms, and/or any other devices and/or components for separating light by wavelengths that may occur to persons skilled in the art upon reviewing the entirety of this disclosure. In some cases, at least a photodetector may be communicative with computing device, such that a sensed signal may be communicated with computing device.

With continued reference to FIG. 12, in some embodiments, microfluidic feature 1208 may include a sensor interface. Sensor interface may be configured to wet waveguide with at least a fluid contained within or otherwise acted upon by microfluidic device 1204. As used in this disclosure, a “sensor interface” is an arrangement permits sensor device 1224 to be in sensed communication with microfluidic device 1204. In some embodiments, sensor interface may include an optical interface. As used in this disclosure, an “optical interface” is an arrangement permits optical device to be in sensed communication with microfluidic device 1204. In one embodiment, sensor device may be coupled to a sensor interface that includes a porous membrane (e.g., nitrocellulose, paper, glass fiber, etc.) as described below that promotes capillary flow. In some cases, a surface of sensor device may be modified with hydrophilic chemistry, for instance by way of silanes, proteins, or another treatment (or may already be hydrophilic) in the sensing region. For example, one or more of sensor devices and sensor interfaces may be configured such that liquid wicks from a porous membrane to a surface of sensor device as it flows through the membrane.

Still referring to FIG. 12, in some embodiments, sensor interface of microfluidic feature 1208 may include a flow cell. As used in this disclosure, a “flow cell” is a component of or associated with a microfluidic device that contains and provides access to a fluid or a flow of a fluid for a sensor interface arrangement. In some cases, a flow cell may effectively increase an area over which at least a fluid flows, thereby increasing access to the at least a fluid for optical sensing. In some cases, a flow cell may include micro-posts. In some cases, a flow cell may include a plurality of micro-posts. As used in this disclosure, “micro-posts” are small scale (e.g., sub-millimeter) protrusions which break up a flow path. In some cases, a micro-post property may be varied in order to affect a flow property. Exemplary non-limiting micro-post properties include pitch, micro-post width (e.g., diameter), micro-post arrangement (e.g., hexagonal), micro-post size (e.g., column), micro-post height, number of micro-posts (total, in a row, in a column, etc.), and the like.

Still referring to FIG. 12, in some embodiments, sensor interface of microfluidic feature 1208 may include a porous membrane. As used in this disclosure, a “porous membrane” is a material with a plurality of voids. In some cases, a porous membrane may have at least a membrane property selected to achieve at least a flow property. As used in this disclosure, a “membrane property” is an objective characteristic associated with a porous membrane. Exemplary non-limiting membrane properties include pore size, porosity, measures of hydrophilicity, measures of surface tension, measures of capillary action, material, and the like. In some embodiments, a porous membrane interfacing with at least a sensor device 1224 and microfluidic device 1204 and/or microfluidic feature 1208 may provide several advantages. In a non-limiting example, a porous membrane connecting two segments of a channel may provide fluidic communication, connecting one segment of the channel to another; (the porous membrane may, thus, carry reagents and/or samples in solution, and open the channel to an outside environment while maintaining fluidic flow to the microfluidic device 1204 and/or microfluidic feature 1208). In another non-limiting example, a porous membrane may eliminate need for a gasket (which may leak and result in poor yield). In a further non-limiting example, a porous membrane may help control one or more flow properties. As used in this disclosure, “flow properties” are characteristics related to a flow of a fluid as described in further detail below in this disclosure. For instance, exemplary non-limiting flow properties include flow rate (in μl/min), flow velocity, integrated flow volume, pressure, differential pressure, and the like. For instance, and without limitation, flow rate within microfluidic feature 1208 may be determined by pore size, pore density, membrane material, and porous membrane dimensions. In other non-limiting examples, a porous membrane strip interfacing at least a sensor device 1224 to microfluidic device 1204 and/or microfluidic feature 1208 may require less precision.

With continued reference to FIG. 12, in some embodiments, microfluidic feature 1208 may include at least a channel. As used in this disclosure, a “channel” is a reservoir having one or more of an inlet (i.e., input) and an outlet (i.e., output). Channels may have a sub millimeter scale consistent with microfluidics. Channels may have channel properties which affect other system properties (e.g., flow properties, flow timing, and the like). As used in this disclosure, “flow timing” is any time-dependent property associated with a flow of at least a fluid. For instance, in some cases, flow timing may include a duration for a flow to reach, pass through, or otherwise interact with an element of microfluidic device 1204 and/or other microfluidic features; for instance, and without limitation, flow out from reservoir 1212. As used in this disclosure, “channel properties” are objective characteristics associated with channels or a microfluidic device generally. Exemplary non-limiting channel properties include width, height, length, material, surface roughness, cross-sectional area, layout, and the like. Additionally, or alternatively, microfluidic feature 1208 may include a microfluidic circuit. As used in this disclosure, a “microfluidic circuit” is a configuration of a plurality of microscale fluidic components within microfluidic device 1204. Microscale fluidic components may include any microfluidic feature 1208 of microfluidic device 1204 as described above. In a non-limiting example, microfluidic circuit may include a configuration of channels, individually addressable valves, and chambers through which fluid is allowed to flow. Microfluidic circuit disclosed here may be consistent with any microfluidic circuit described in U.S. patent application Ser. No. 18/107,135, filed on Feb. 8, 2023, entitled “APPARATUS AND METHODS FOR ACTUATING FLUIDS IN A BIO SENSOR CARTRIDGE,” the entirety of which is incorporated herein by reference.

With continued reference to FIG. 12, microfluidic device 1204 with integrated sensor device 1224 may be utilized in an advanced diagnostic device or diagnostic sensor for detection of biological signatures (e.g., viruses, bacteria, pathogens, and the like). In some cases, microfluidic feature 1208 may be fabricated on a substrate. Substrate may be composed of various materials, such as glass, silicon, and the like. In one or more embodiments, microfluidic device 1204 containing microfluidic features may be fabricated using various processes, such as, for example, photolithography, injection molding, stamping processes, and the like. In various embodiments, substrate may be substantially planar. In some embodiments, microfluidic feature 1208 may be built on a substrate using, for example, photosensitive polymers or photoresists (e.g., SU-8, Ostemer, and the like). In other embodiments, microfluidic feature 1208 may be molded or stamped into polymers (e.g., PMMA). In other embodiments, components and/or devices of microfluidic device 1204 may be built into or on substrate using etching processes, in which channels, reservoir 1212, capillary pumps, and valves may be built by removing materials from substrate. In non-limiting embodiments, the entire microfluidic system may be fabricated on substrate, sealed with a cover plate, where holes are drilled and aligned with certain microfluidic components, such as reservoir 1212. Additionally, or alternatively, substrate may then be diced into small chips. Chips may also be fabricated with microfluidic features etch or patterned on them. Further, they can be coupled to microfluidic features fabricated separately on another substrate such as plastic or glass.

With continued reference to FIG. 12, apparatus 1200 further includes at least a flow component 1228 connected with at least a microfluidic feature 1208 configured to flow at least a fluid through at least a sensor device 1224. In some embodiments, at least a flow component 1228 may include a passive flow component configured to initiate a passive flow process. As used in this disclosure, a “passive flow component” is a component, typically of a microfluidic device, that imparts a passive flow on a fluid, wherein the “passive flow,” for the purpose of this disclosure, is flow of fluid, which is induced absent any external actuators, fields, or power sources. As used in this disclosure, a “passive flow process” is a plurality of actions or steps taken on passive flow component in order to impart a passive flow on at least a fluid. Passive flow component may employ one or more passive flow techniques in order to initiate passive flow process; for instance, and without limitation, passive flow techniques may include osmosis, capillary action, surface tension, pressure, gravity-driven flow, hydrostatic flow, vacuums, and the like. Passive flow component may be in fluidic communication with at least a reservoir 1212. Exemplary non-limiting passive flow component is explained in greater detail in this disclosure below. Passive flow component may be configured to flow at least a fluid stored in at least a reservoir 1212 with predetermined flow properties. In a non-limiting example, passive flow component may be consistent with any passive flow component described in U.S. patent application Ser. No. 17/859,932, filed on Jul. 7, 2022, entitled “SYSTEM AND METHODS FOR FLUID SENSING USING PASSIVE FLOW,” the entirety of which is incorporated herein by reference.

With continued reference to FIG. 12, in other embodiments, at least a flow component 1228 may include an active flow component configured to initiate an active flow process. As used in this disclosure, an “active flow component” is a component that imparts an active flow on a fluid, wherein the “active flow,” for the purpose of this disclosure, is flow of fluid which is induced by external actuators, fields, or power sources. As used in this disclosure, an “active flow process” is a plurality of actions or steps taken on active flow component in order to impart active flow on at least a fluid. In some embodiments, active flow component 1216 is in fluidic communication with at least a reservoir 1212. In a non-limiting example, active flow component may include one or more pumps. Pump may include a substantially constant pressure pump (e.g., centrifugal pump) or a substantially constant flow pump (e.g., positive displacement pump, gear pump, and the like). Pump can be hydrostatic or hydrodynamic. As used in this disclosure, a “pump” is a mechanical source of power that converts mechanical power into fluidic energy. A pump may generate flow with enough power to overcome pressure induced by a load at a pump outlet. A pump may generate a vacuum at a pump inlet, thereby forcing fluid from a reservoir into the pump inlet to the pump and by mechanical action delivering this fluid to a pump outlet. Hydrostatic pumps are positive displacement pumps. Hydrodynamic pumps can be fixed displacement pumps, in which displacement may not be adjusted, or variable displacement pumps, in which the displacement may be adjusted. Exemplary non-limiting pumps include gear pumps, rotary vane pumps, screw pumps, bent axis pumps, inline axial piston pumps, radial piston pumps, and the like. Pump may be powered by any rotational mechanical work source, for example without limitation and electric motor or a power take off from an engine. Pump may be in fluidic communication with at least a reservoir 1212. In some cases, reservoir 1212 may be unpressurized and/or vented. Alternatively, reservoir 1212 may be pressurized and/or sealed; for instance, by alignment component 1216 such as, without limitation, sealer as described above. In a non-limiting example, active flow component may include any active flow component as described in U.S. patent application Ser. No. 18/107,135. Exemplary non-limiting active flow component is explained in greater detail in this disclosure below in reference to FIGS. 10A-B.

With continued reference to FIG. 12, in some cases, development of microfluid feature layout, selection of flow component, and sensor interface may need to be performed in an iterative design process as each parameter is interdependent with important system properties (e.g., flow properties and flow timing). In some embodiments, aspect ratios of chambers (e.g., reservoir 1212), fluidic resistances (controlled by dimensions) of channels between the chambers (and sensor interface), and flow component parameters (e.g., pump pressure) may be tuned to affect one or both of timing and flow of at least a fluid. In some embodiments, microfluidic feature 1208 within microfluidic device 1204 of apparatus 1200 may be hydrophilic, for example through coating, to ensure flow. Alternatively, or additionally, microfluidic device 1204 may include a hydrophilic material, such as without limitation polymethyl methacrylate (PPMA). Further, a reagent chamber may be placed such that the sensor reaction chamber is between the reagent chamber and the sample chamber.

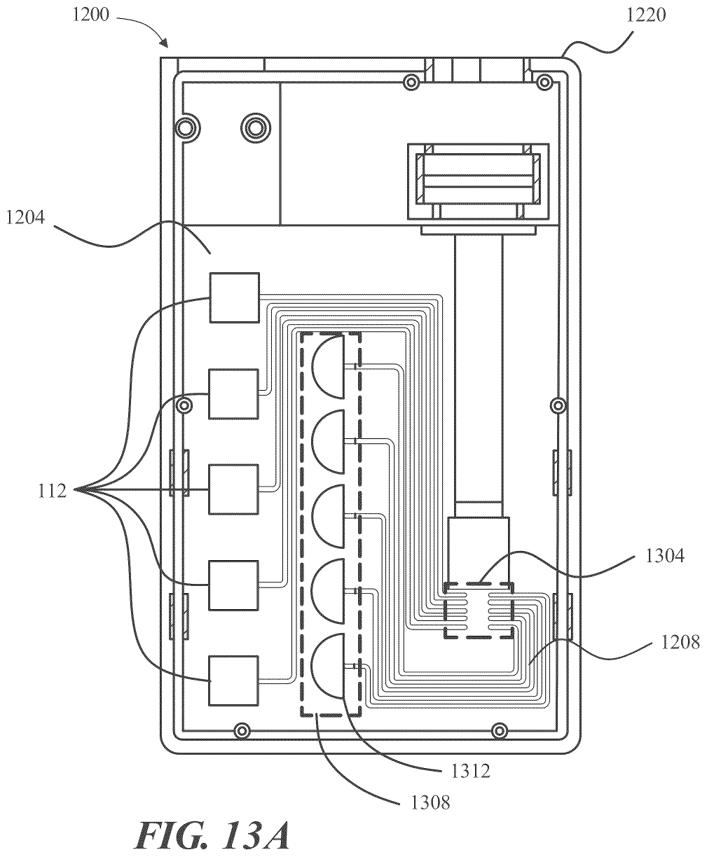

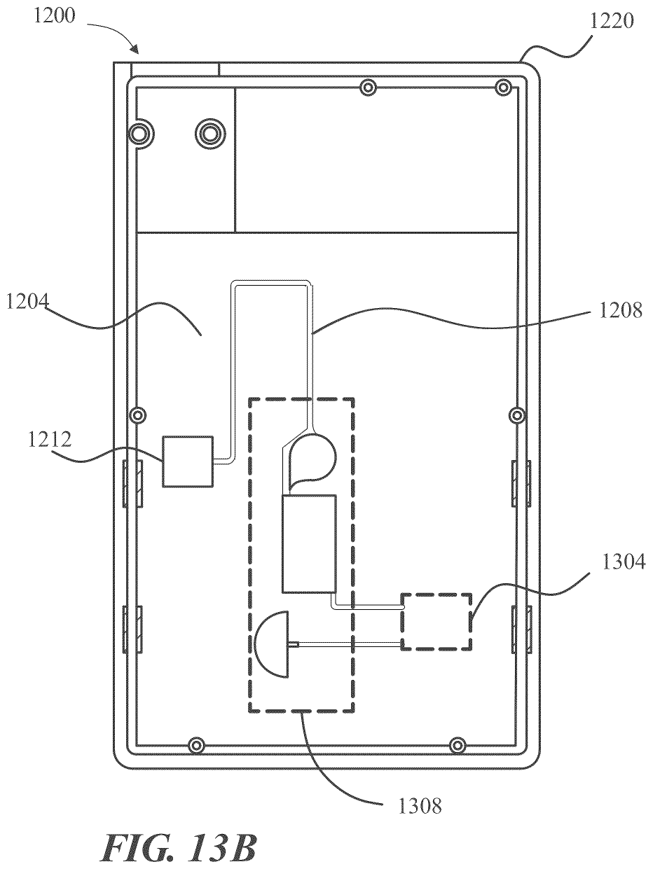

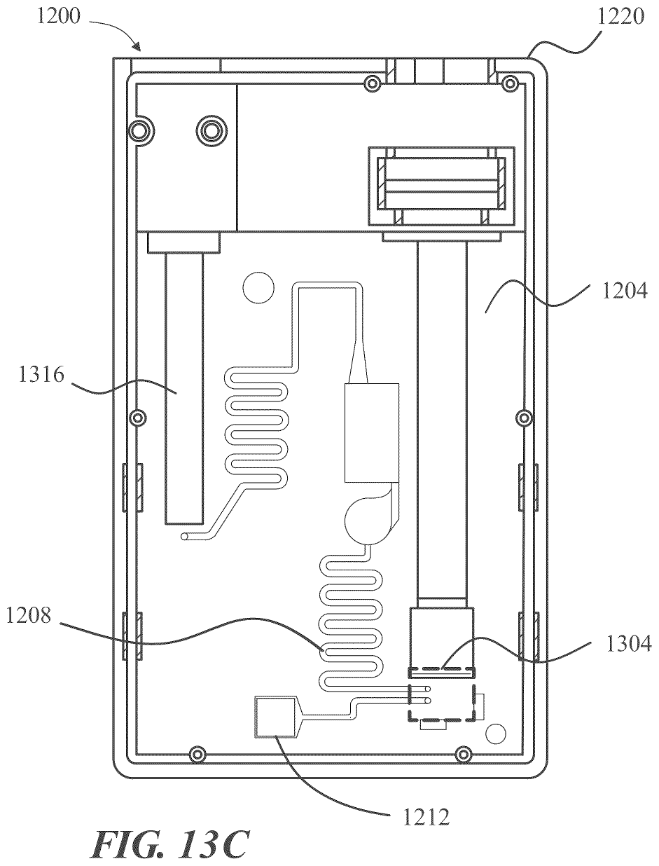

Now referring to FIGS. 13A-C, exemplary embodiments of at least a sensor device 1224 integrated to different microfluidic environments are illustrated. At least a sensor device 1224 may be disposed in a sensor area 1304. As used in this disclosure, a “sensor area” is a position, a location, or otherwise an area determined by at least an alignment feature 1216 as described above. In some embodiments, sensor area may match with at least a surface of sensor device 1224; for instance, and without limitation, alignment feature 1216 may include a slightly depressed plane, wherein the slightly depressed plane may include a same surface area with the at least a surface of sensor device 1224. In some embodiments, microfluidic feature 1208 may be configured to pass through sensor area 1304. In some cases, microfluidic feature 1208 such as microfluidic channels may pass underneath sensor area 1304. In other cases, microfluidic feature 1208 such as microfluidic channels may pass above sensor area 1304. In a non-limiting example, sensor area 1304 may be located at a first layer, wherein the first layer may be above or below a second layer containing the microfluidic environment. At least an alignment feature 1216, such as, without limitation, a sealer, may be placed between the first layer and the second layer; however, the sealer may avoid at least a portion of sensor area 1304 in order for sensor device 1224 disposed at sensor area 1304 to detect sensed properties as described above, such as, without limitation, optical properties (e.g., wavelength, frequency, intensity, polarization, spectral distribution, absorption and emission spectra, and the like) and flow properties (e.g., flow rate, flow velocity, integrated flow volume, pressure, and the like). As used in this disclosure, a “microfluidic environment” refers to a complex system of plurality of microfluidic features such as, without limitation, microfluidic channels, chambers, valves, other components within microfluidic devices 1204 that are used to transport and/or manipulate at least a fluid on a microscale within apparatus 1200.

Still referring to FIGS. 13A-C, exemplary embodiments of at least a sensor device 1224 integrated to a passive microfluidic environment is illustrated. In some embodiments, microfluidic environment may include a passive microfluidic environment, wherein the passive microfluidic environment is a microfluidic environment driven by passive flow component 1308. Passive flow component 1308 may include any passive flow component as described in this disclosure. In a non-limiting example, flow of at least a fluid within passive microfluidic environment may only include passive flow. Passive microfluidic environment may utilize capillary action or wicking, provided by passive flow component 1308, to flow at least a fluid through microfluidic feature 1208 of microfluidic device 1204 as described above. In some embodiments, passive flow component 1308 may include a capillary pump 1312. As used in this disclosure, a “capillary pump” is a component that operates without any external power source and relies on capillary action to move at least a fluids in fluidic communication with the capillary pump 1312. “Capillary action,” for the purpose of this disclosure, is a phenomenon that occurs when a liquid such as, without limitation, at least a fluid, in contact with a solid surface such as, without limitation, sensor interface including porous membrane, and is able to move against gravity due to the combined effects of adhesive and cohesive forces. In a non-limiting example, passive flow process may be initiated as a function of such capillary action. In a non-limiting example, when the porous membrane is in contact with the at least a fluid in a first reservoir, the at least a fluid may be drawn into the pores of the porous membrane due to capillary action. First reservoir may be located at a first layer. A pressure difference may be created across the medium as the at least a fluid fills the pores; for instance, and without limitation, pressure may be higher on the side of the porous membrane that is in contact with the at least a fluid. Such pressure difference may cause the at least a fluid to flow through the sensor interface and into a second reservoir, wherein the second reservoir may be located at a second layer, and wherein the first layer is above the second layer, separated by sealer. In some cases, capillary pump 1312 may operate continuously, as long as there is a sufficient supply of fluid in first reservoir. Flow properties such as, without limitation, the rate of flow of at least a fluid through capillary pump 1312 may be determined by the size and porosity of the porous membrane, the surface tension of the at least a fluid, and the height difference between first reservoir and second reservoir. Additionally, or alternatively, passive microfluidic environment (as shown in FIG. 2B) may utilize other microfluidic feature such as, without limitation, a conjugate pad, and a bubble trap, wherein both component will be described in further detail below. Further, in other embodiments, microfluidic environment may include an active microfluidic environment (as shown in FIG. 2C), wherein the active microfluidic environment is a microfluidic environment driven by active flow component 1316. Active flow component may include any active flow component as described in this disclosure. Elements of active flow component 1316 are described in further detail below in this disclosure. In such embodiment, active microfluidic environment may utilize a pressure, produced and/or varied by active flow component 1316 powered by a power source, to flow at least a fluid through microfluidic feature 1208 of microfluidic device 1204.

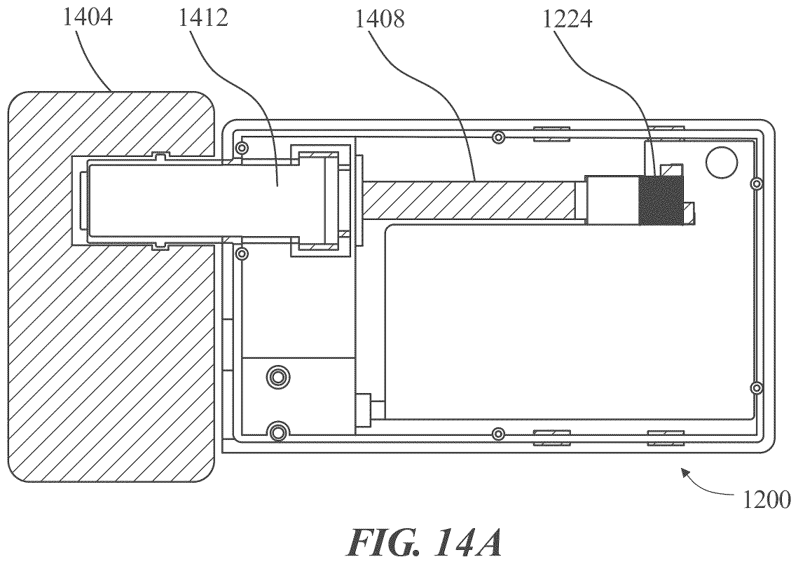

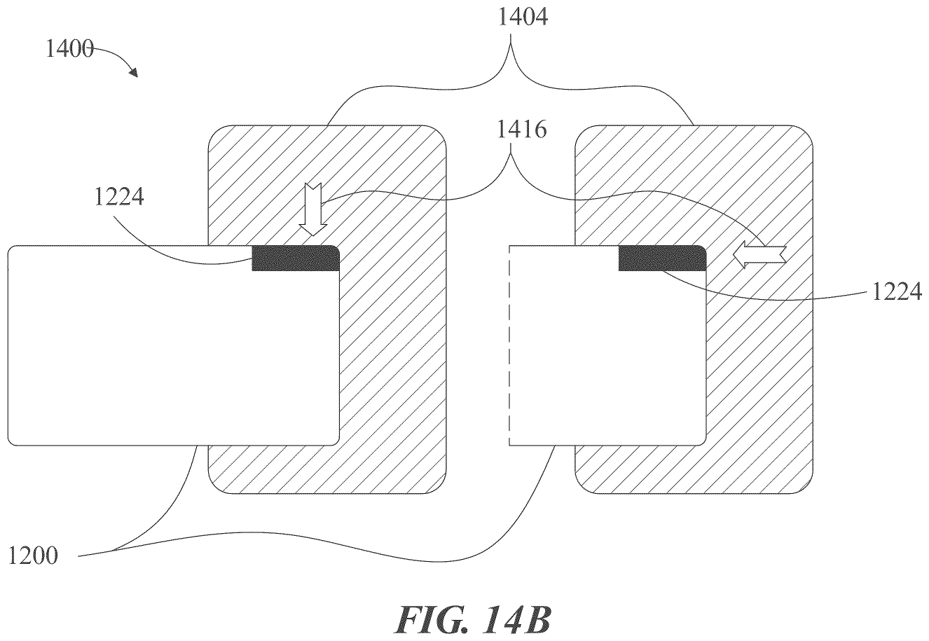

Now referring to FIGS. 14A-B, exemplary embodiments of possible data transfer from apparatus 1200 to external device 1404 are illustrated. As used in this disclosure, an “external device” generally refers to any device or component that is physically separate from apparatus 1200 from the exterior. In some embodiments, external device may include any computing device as described in this disclosure. External device 1404 may include a device such as, without limitation, a computing device as described in this disclosure, that is not an integral part of apparatus 1200 but is instead connected or interfaced with apparatus 1200 in some way to provide additional functionality or capabilities. In a non-limiting example, external device 1404 may include an external reader configured read and/or process sensed properties from sensor device 1224 as described above. External device 1404 may be configured to read, interpret, or otherwise record (optical, electrical, and/or magnetic) signals generated and output by sensor device 1224. In some embodiments, external device 1404 may be used in conjunction with apparatus 1200 for performing microfluidic-based biochemical assay. In a non-limiting example, sensor device 1224 may transfer output data containing, without limitation, sensed properties to external device 1404 using an optical fiber ribbon 1408 and a multi-fiber push connector (MPO) 1412 (as shown in FIG. 5A). An “optical fiber ribbon,” for the purpose of this disclosure, is a specialized cable consisting of a plurality of optical fibers bundled together in a flat, ribbon-like configuration. Each optical fibers of plurality of optical fibers may be made of glass or plastic. Each optical fibers of plurality of optical fibers may be configured to transmit light signals with very low loss over a long distances. In some embodiments, optical fiber ribbon may be used to transfer optical properties as described above from sensor device 1224 to external device 1404 through MPO 1412. As used in this disclosure, a “multi-fiber push connector” is a connection component configured to connect optical fiber ribbon between apparatus 1200 and external device 1404. In some embodiments, MPO 1412 may include a plug with a row of plurality of optical fibers that are aligned and held in place by precision pins. The connector typically consists of a plug with a row of plurality of optical fibers that are aligned and held in place by one or more precision pins, wherein the precision pins are pins used in manufacturing and assembly processes to ensure precise and accurate alignment of components such as, without limitation, plurality of optical fibers.

Still referring to FIGS. 14A-B, Additionally, or alternatively, connection between apparatus 1200 and external device 1404 may be accomplished via laser coupling process 1400 (as shown in FIG. 5B). As used in this disclosure, a “laser coupling process” refers to a process of optically connecting or coupling external device 1404 to sensor device 1224 via light source 1416. Light source 1416 may include any light source as described in this disclosure. Data transfer between sensor device 1224 and external device 1404 via laser coupling process 1400 may be referred to as a fibreless data transfer. In some embodiments, laser coupling 1400 may be performed using one or more lens assemblies which are designed to precisely align laser beam with the input of the receiving component such as, without limitation, external device 1404. The lens assembly may be used to collimate or focus the laser beam. In case of external device 1404 containing light source 1416, receiving component may include apparatus 1200. In a non-limiting example, transferring data between external device 1404 and apparatus 1200 may include configuring light source 1416 within external device 1404 to irradiate a laser at sensor device 1224. In some embodiments, light source 1416 may include different orientations relative to connected component in laser coupling process 1400. In a non-limiting example, laser coupling process 1400 may include a horizontal laser coupling, wherein the horizontal laser coupling may include a horizontal orientation of light source and external device 1404. In such embodiment, laser beam may be directed horizontally from lights source into the input of external device 1404. In another non-limiting example, laser coupling process 1400 may include a vertical (or edge) laser coupling, wherein the vertical laser coupling may include a vertical orientation of light source and external device 1404. In such embodiment, laser beam may be directed vertically from light source into the input of external device 1404. In a further embodiment, horizontal laser coupling and vertical laser coupling may be used in combination to achieve an optimal performance such as, without limitation, external device 1404 may be configured to receive and/or transmit signals in multiple directions.

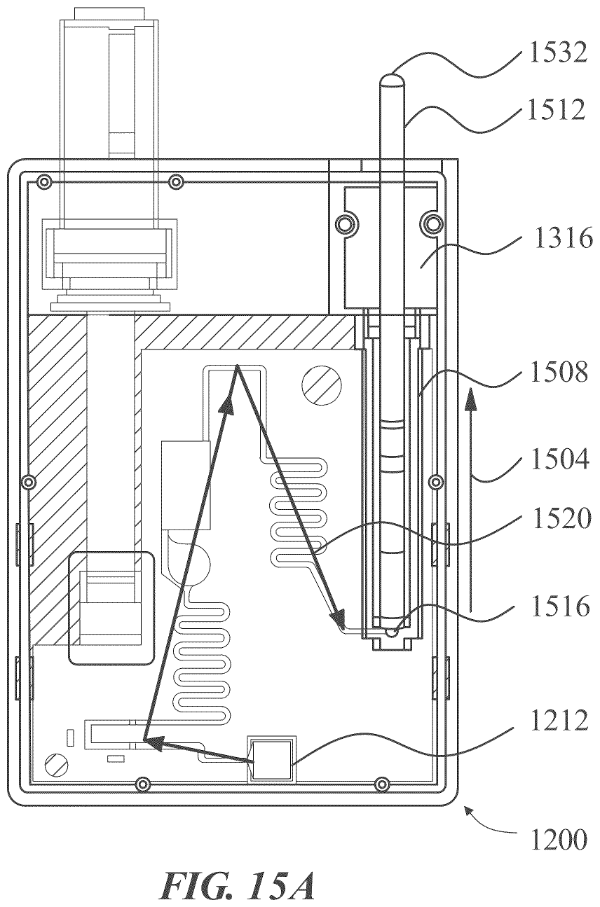

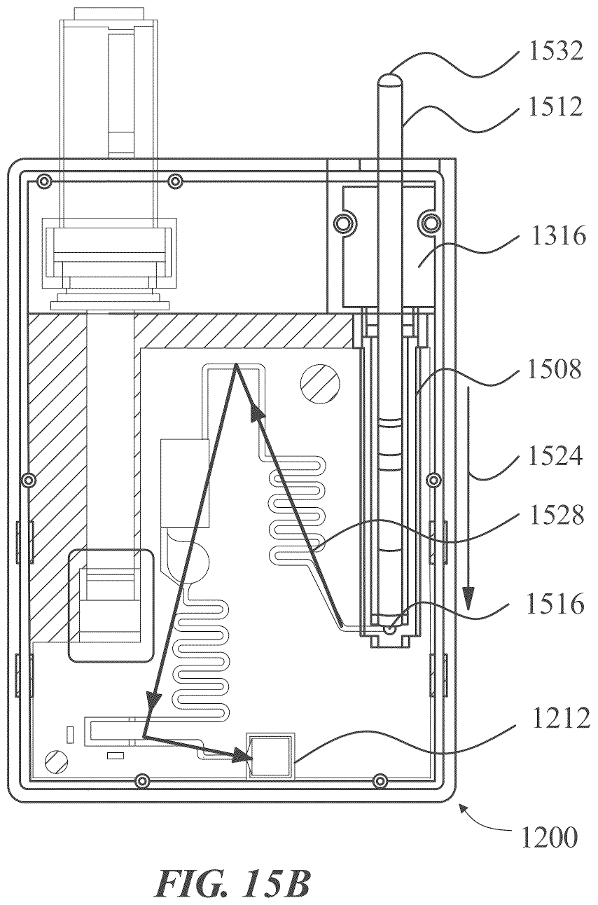

With continued reference to FIG. 15A-B, pull regime 1504 and/or push regime 1524 of active flow component 1316 may be driven by an actuator, wherein the actuator may be connected to plunger 1512 through a mechanical interface 1532. As used in this disclosure, an “actuator” is a device that produces a motion by converting energy and signals going into the system. In some cases, motion may include linear, rotatory, or oscillatory motion. Actuator may include a component of a machine that is responsible for moving and/or controlling a mechanism or system. Actuator may, in some cases, require a control signal and/or a source of energy or power. In some cases, a control signal may be relatively low energy. Exemplary control signal forms include electric potential or current, pneumatic pressure or flow, or hydraulic fluid pressure or flow, mechanical force/torque or velocity, or even human power. In some cases, an actuator may have an energy or power source other than control signal. This may include a main energy source, which may include for example electric power, hydraulic power, pneumatic power, mechanical power, and the like. In some cases, upon receiving a control signal, actuator responds by converting source power into mechanical motion. In some cases, actuator may be understood as a form of automation or automatic control. Additionally, or alternatively, actuator may be enclosed by housing 1220. In such embodiment, housing 1220 may protect actuator from damage by external factors.

With continued reference to FIG. 15A-B, in some embodiments, actuator may include a hydraulic actuator. A hydraulic actuator may consist of a cylinder or fluid motor that uses hydraulic power to facilitate mechanical operation. Output of hydraulic actuator may include mechanical motion as described above. In some cases, hydraulic actuator may employ a liquid hydraulic fluid. As liquids, in some cases. are incompressible, a hydraulic actuator can exert large forces. Additionally, as force is equal to pressure multiplied by area, hydraulic actuators may act as force transformers with changes in area (e.g., cross sectional area of cylinder and/or piston). An exemplary hydraulic cylinder may consist of a hollow cylindrical tube within which a piston can slide. In some cases, a hydraulic cylinder may be considered single acting. Single acting may be used when fluid pressure is applied substantially to just one side of a piston. Consequently, a single acting piston can move in only one direction. In some cases, a spring may be used to give a single acting piston a return stroke. In some cases, a hydraulic cylinder may be double acting. Double acting may be used when pressure is applied substantially on each side of a piston; any difference in resultant force between the two sides of the piston causes the piston to move.

With continued reference to FIG. 15A-B, in some embodiments, actuator may include a pneumatic actuator. In some cases, a pneumatic actuator may enable considerable forces to be produced from relatively small changes in gas pressure. In some cases, a pneumatic actuator may respond more quickly than other types of actuators, for example hydraulic actuators. A pneumatic actuator may use compressible fluid (e.g., air). In some cases, a pneumatic actuator may operate on compressed air. Operation of hydraulic and/or pneumatic actuators may include control of one or more valves, circuits, fluid pumps, and/or fluid manifolds.

With continued reference to FIG. 15A-B, in some cases, actuator may include an electric actuator. Electric actuator may include any of electromechanical actuators, linear motors, and the like. In some cases, actuator may include an electromechanical actuator. An electromechanical actuator may convert a rotational force of an electric rotary motor into a linear movement to generate a linear movement through a mechanism. Exemplary mechanisms, include rotational to translational motion transformers, such as without limitation a belt, a screw, a crank, a cam, a linkage, a scotch yoke, and the like. In some cases, control of an electromechanical actuator may include control of electric motor, for instance a control signal may control one or more electric motor parameters to control electromechanical actuator. Exemplary non-limitation electric motor parameters include rotational position, input torque, velocity, current, and potential. electric actuator may include a linear motor. Linear motors may differ from electromechanical actuators, as power from linear motors is output directly as translational motion, rather than output as rotational motion and converted to translational motion. In some cases, a linear motor may cause lower friction losses than other devices. Linear motors may be further specified into at least 3 different categories, including flat linear motor, U-channel linear motors and tubular linear motors. Linear motors may be directly controlled by a control signal for controlling one or more linear motor parameters. Exemplary linear motor parameters include without limitation position, force, velocity, potential, and current.

With continued reference to FIG. 15A-B, in some embodiments, actuator may include a mechanical actuator. In some cases, a mechanical actuator may function to execute movement by converting one kind of motion, such as rotary motion, into another kind, such as linear motion. An exemplary mechanical actuator includes a rack and pinion. In some cases, a mechanical power source, such as a power take off may serve as power source for a mechanical actuator. Mechanical actuators may employ any number of mechanism, including for example without limitation gears, rails, pulleys, cables, linkages, and the like. In a non-limiting example, actuator may include a linear actuator. As used in this disclosure, a “linear actuator” is an actuator that creates linear motion. Linear actuator may create motion in a straight line; for instance, and without limitation, active flow component 216, particularly, plunger 1512 and/or barrel 1508 may be aligned with the straight line. Pull regime 1504 and/or push regime 1524 may be driven by such linear actuator. Actuator may include any actuator described in U.S. patent application Ser. No. 18/107,135.

With continued reference to FIG. 15A-B, a “mechanical interface,” for the purpose of this disclosure, is a component configured to connect at least two components. In an embodiment, mechanical interface 1532 between plunger 1512 and actuator may be a friction fit, an interference fit, or a snap fit, wherein plunger 1512 may include a male or female adapter and the actuator 1220 may include a female or male adapter. For example, and without limitation, when the male (female) adapter engages with the female (male) adapter a mechanical connection is established. This mechanical connection can be designed so that it automatically disengages when a certain level of force is applied. Alternatively, it can be designed so that a mechanical input is necessary to cause the male and female connectors to disengage. In some embodiments, this mechanical coupling between plunger 1512 and actuator may be accomplished by other means (e.g., a Janney coupler, knuckle coupler, etc.). In other embodiments, mechanical coupling between plunger 1512 and actuator may be accomplished by a magnet or multiple magnets.

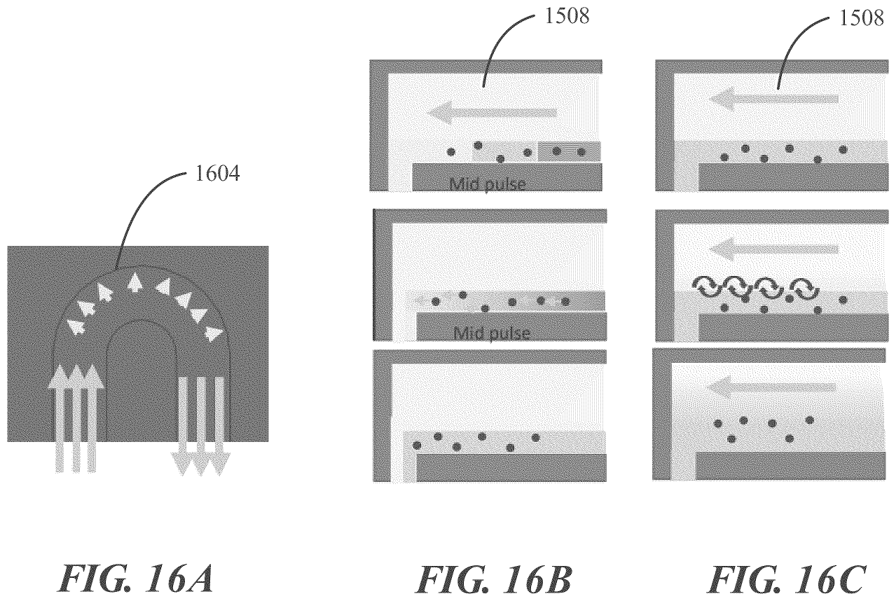

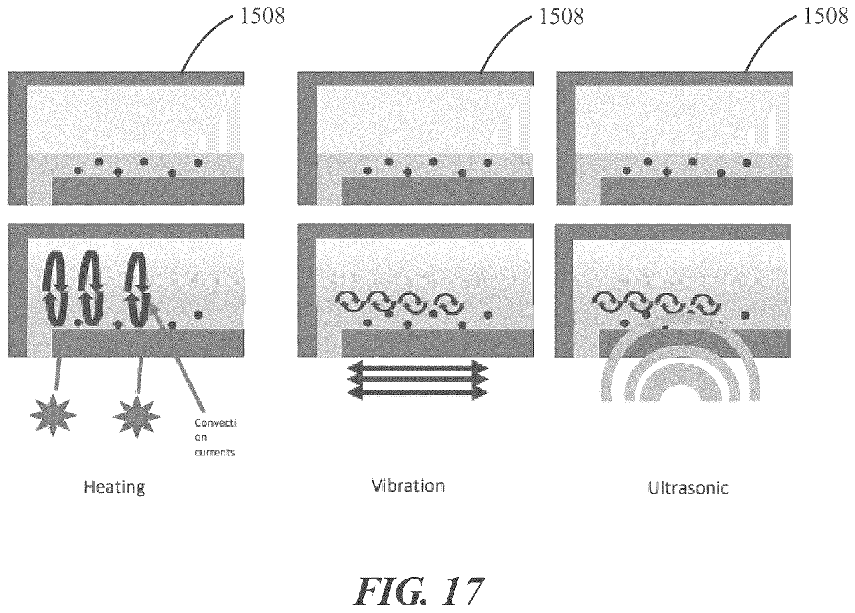

Now referring to FIG. 16A-C, exemplary embodiments of plurality of microfluidic features 1208 that may be utilized for both lateral and longitudinal mixing are illustrated. In some embodiments, flow component 1228 may be configured to mix a first fluid with a second fluid, wherein the first fluid may include a sample fluid with a conjugate reagent, applied by conjugate pad 1408 as described above, and the second fluid may include a buffer fluid. Mixing of the first fluid and the second fluid may occur during either reverse flow process 1520 or forward flow process 1528. In some embodiments, at least a microfluidic features 1208 may include one or more serpentines 1604 (as shown in FIG. 16A), wherein the “serpentine,” for the purpose of this disclosure, refers to a specific configuration or pattern of channels in microfluidic device 1204. In some embodiments, serpentine 1604 may be configured to provide a large surface area for fluid mixing. In other embodiments, serpentine 1604 may be configured to increase the residence time of fluids within microfluidic features 1208. In some cases, serpentines 1604 may be formed by a series of interconnected loops or meanders that create a tortuous path for fluids to flow through. Serpentines 1604 may be designed and/or optimized to achieve desired fluid property such as, without limitation, mixing efficiency, reaction kinetics, separation performance, and the like thereof. In a non-limiting example, serpentines 1604 may be used for lateral mixing, wherein the “lateral mixing,” as described herein, is a process for achieving uniform distribution of one or more fluids by mixing fluids horizontally. Additionally, or alternatively, mixing first fluid with second fluid may further include mixing first fluid with second fluid as a function of a longitudinal mixing in flow component 1228, wherein the “longitudinal mixing,” as described herein, is a process for achieving uniform distribution of one or more fluids by mixing fluids vertically. In a non-limiting example, longitudinal mixing may occur within barrel 1508, wherein rapid flow pulses may cause conjugates to move turbulently due to inertia (as shown in FIG. 16B) and interfacial friction (as shown in FIG. Other means of mixing are described in further detail with reference to FIG. 17.

Now referring to FIG. 17, an exemplary embodiment of other types of mixing using flow component 1228 is illustrated. In an embodiment, active flow component 1316 may be configured to mix first fluid and second fluid as described above. In such embodiment, mixing such as, without limitation, longitude mixing may occur within barrel 1508 of active flow component 1316. In a non-limiting example, longitude mixing may include mixing via heat (as shown on the left of FIG. 17). In another non-limiting example, longitude mixing may include mixing via vibration (as shown in the middle of FIG. 17). In a further non-limiting example, longitude mixing may include mixing via ultrasonic. In other non-limiting example, longitude mixing may further include mixing via electromagnetic. Person skilled in the art, upon reviewing the entirety of this disclosure, will be aware of various type of mixing fluids through flow component 1228 that may be used for performing microfluidic-based biochemical assays.

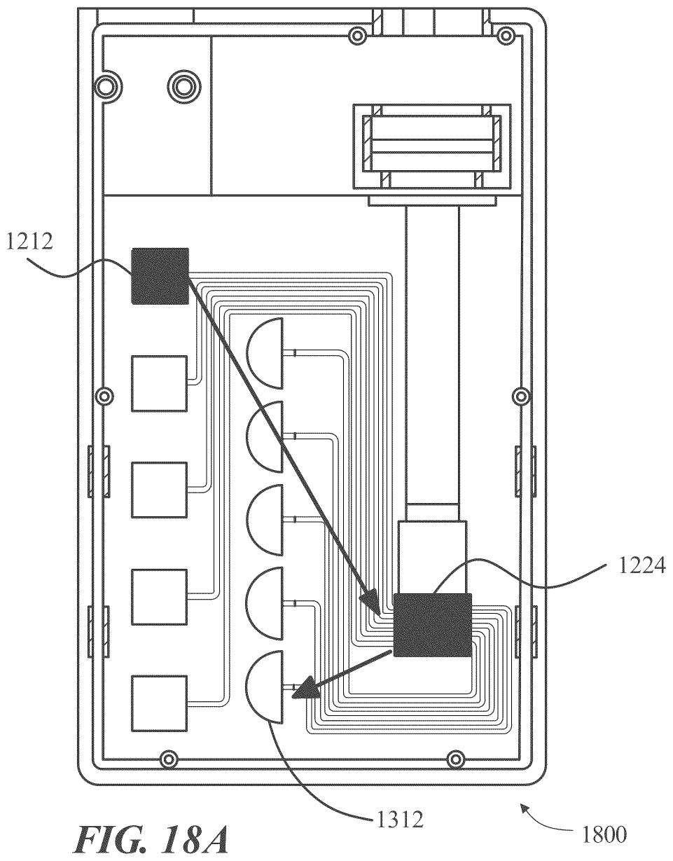

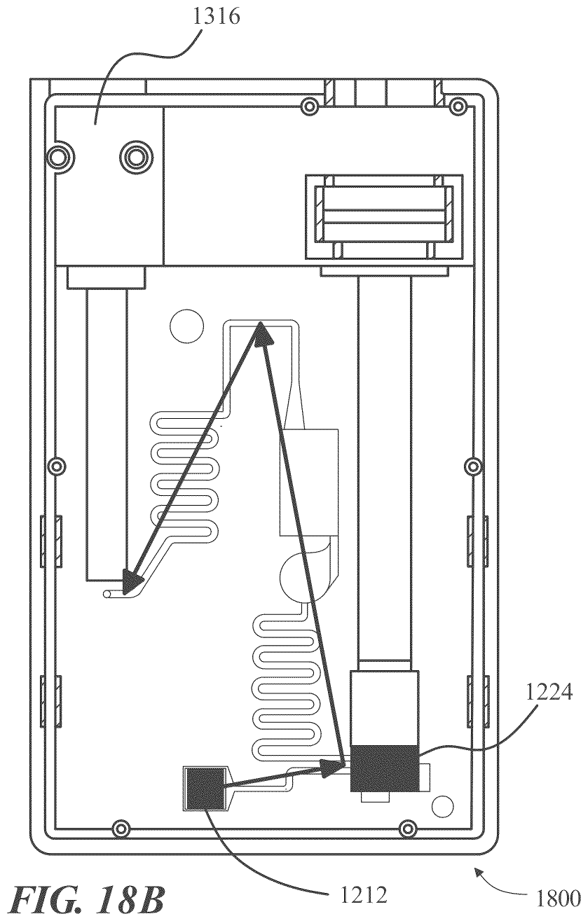

Now referring to FIG. 18A-B, exemplary embodiments of single step assay 1800 performed using apparatus 1200 are illustrated. In a non-limiting example, a pre-mixed mixture of reagents and sample may be added to reservoir 1212. The mixture then flows through the sensor device 1224 driven by a capillary liquid movement driven by a capillary pump 1312 such as, without limitation, a wicking effect of a wicking paper at the end of microfluidic feature 1208 (as shown in FIG. 18A) or pull regime 1504 of active flow component 1316 (as shown in FIG. 18B).

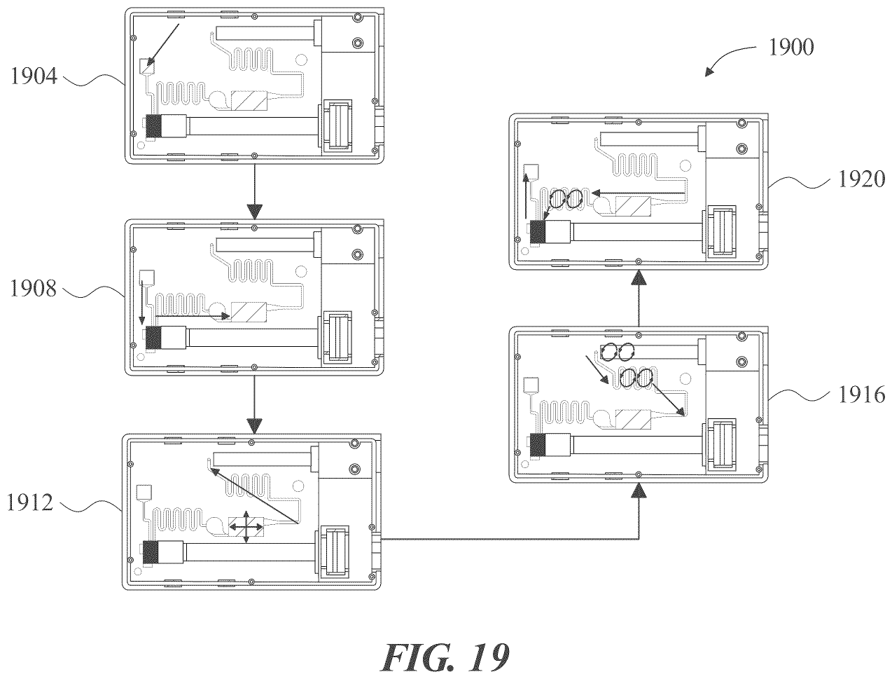

Now referring to FIG. 19, an exemplary embodiment of a two-step assay 1900 performed using apparatus 1200 is illustrated. Two-step assay 1900 may include a step 1904 of adding a sample into reservoir 1212. Two-step assay 1900 may include a step 1908 of flowing the sample to conjugate pad 1408 as a function of a reverse flow process initiated by active flow component 1316 using pull regime 1504. Two-step assay 1900 may include a step 1912 of releasing a conjugate regent stored in conjugate pad 1408. Two-step assay 1900 may include a step of 1916 of flowing the sample and conjugate regent as a function of forward flow process initiated by active flow component 1316 using push regime 1524, wherein flowing the sample and conjugate regent may further include mixing conjugate regent and sample within barrel 1508 of active flow component 1316 and/or microfluidic feature 1208 after and/or before conjugate pad 1408. Two-step assay 1900 may further include a step 1920 of flowing the mixture of sample and conjugate regent through sensor device 1224 and back to reservoir 1212.

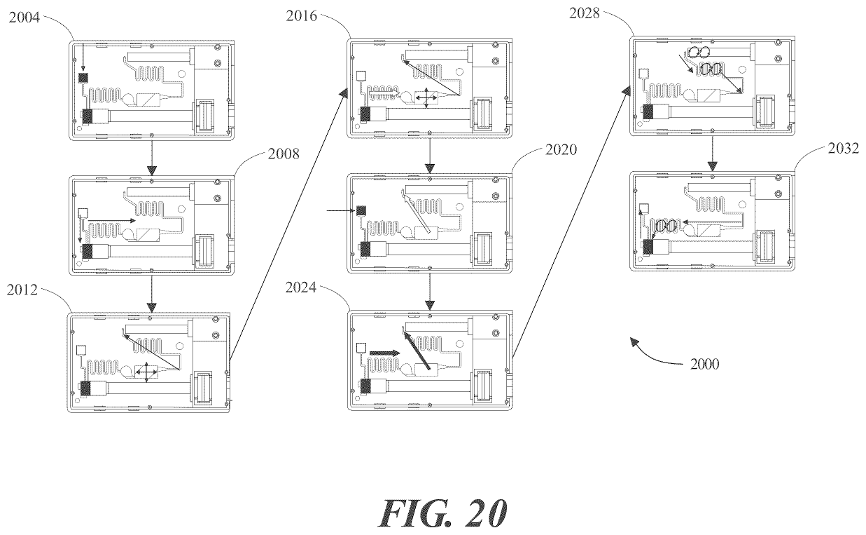

Now referring to FIG. 20, an exemplary embodiment of a three-step assay 2000 performed using apparatus 1200 is illustrated. Three-step assay 2000 may include a step 2004 of adding a sample into reservoir 1212. Three-step assay 2000 may include a step 2008 of flowing the sample to conjugate pad 1408 as a function of a reverse flow process initiated by active flow component 1316 using pull regime 1504. Three-step assay 2000 may include a step 2012 of releasing a conjugate regent stored in conjugate pad 1408. Three-step assay 2000 may include a step 2016 of adding a buffer fluid, driven by pull regime 1504. Three-step assay 2000 may include a step 2020 of receiving buffer fluids, sample, and conjugate regent at active flow component 1316. Three-step assay 2000 may include a step 2024 of utilizing an air bubble as a separation barrier between buffer fluid and any regents added later. Three-step assay 2000 may include a step 2028 of flowing received fluids (i.e., pre-mixed regents, conjugate reagent, and sample buffer) as a function of forward flow process initiated by active flow component 1316 using push regime 1524, wherein flowing the fluids may further include mixing fluids within barrel 1508 of active flow component 1316 and/or microfluidic features 1208 after and/or before conjugate pad 1408. Three-step assay 2000 may further include a step 2032 of flowing the mixture through sensor device 1224 and back to reservoir 1212.

IgE may be detected using a device consistent with a device disclosed in U.S. patent application Ser. No. 18/107,135, filed on Feb. 8, 2023, and titled “APPARATUS AND METHODS FOR ACTUATING FLUIDS IN A BIOSENSOR CARTRIDGE,” U.S. patent application Ser. No. 18/121,712, filed on Mar. 15, 2023, and titled “APPARATUS AND METHODS FOR PERFORMING MICROFLUIDIC-BASED BIOCHEMICAL ASSAYS,” and/or U.S. patent application Ser. No. U.S. patent application Ser. No. 18/126,014, filed on Mar. 24, 2023, and titled “PHOTONIC BIOSENSORS FOR MULTIPLEXED DIAGNOSTICS AND A METHOD OF USE,” each of which is incorporated in its entirety by reference.

Antibodies may be produced by any method in the art, e.g., protein synthesis, recombinant techniques, etc.

Recombinant techniques may be used for production of antibodies. In a non-limiting example, nucleic acids encoding light and heavy chain variable regions, optionally linked to constant regions, may be inserted into expression vectors. Light and heavy chains may be cloned in the same or different expression vectors. DNA segments encoding immunoglobulin chains may be operably linked to control sequences in expression vector(s) that ensure the expression of immunoglobulin polypeptides. Expression control sequences may include promoters (e.g., naturally associated or heterologous promoters), signal sequences, enhancer elements, and transcription termination sequences. An expression control sequence may include a eukaryotic promoter system in a vector capable of transforming or transfecting eukaryotic host cells (e.g., COS or CHO cells). Once a vector has been incorporated into an appropriate host, the host may be maintained under conditions suitable for high level expression of the nucleotide sequences, and the collection and purification of antibodies.

In some embodiments, antibodies according to the present invention may include single chain polypeptides, and can be synthesized using chemical peptide synthesis methods in the art. In a non-limiting example, a single chain antibody may be synthesized using solid phase synthesis.

Once synthesized, an antibody may be purified using, in non-limiting examples, ammonium sulfate precipitation, affinity columns, column chromatography, high performance liquid chromatography (HPLC) purification, gel electrophoresis, and the like.

In some embodiments, disclosed herein is a kit that includes one or more IgG binders and/or one or more IgG binder aggregators. Such kits may be useful, for example, in carrying out a method according to the present invention. In some embodiments, a kit may include one or more of: one or more IgG binders, one or more IgG binder aggregators, and/or one or more anti-IgE antibody nanoparticle conjugates. In some embodiments, a kit may include one or more of: a nucleic acid molecule encoding an IgG binder and/or an IgG binder aggregator, and a cell comprising such a nucleic acid. In some embodiments, a kit may further include a biolayer interferometry system, a component for collecting and/or storing a subject sample, and an instruction manual.

In some embodiments, a method described herein may further include determining whether a subject has an allergy. In a non-limiting example, a subject sample may be collected, IgG in the sample may be depleted, the sample may be tested for levels of IgE antibodies specific to one or more antigens, and a decision may be made as to whether the subject has an allergy.

In some embodiments, a method described herein may further include determining whether a subject is in need of a treatment. In a non-limiting example, a subject sample may be collected, IgG in the sample may be depleted, the sample may be tested for levels of IgE antibodies specific to one or more antigens, and a decision may be made as to whether the subject is in need of an allergy related treatment. In some embodiments, a method may further include administering a treatment to a subject.

As used herein, the singular forms “a,” “an,” and “the” designate both the singular and the plural, unless expressly stated to designate the singular only.

As used herein, the term “about” and the use of ranges in general, whether or not qualified by the term about, means that the number comprehended is not limited to the exact number set forth herein, and is intended to refer to ranges substantially within the quoted range while not departing from the scope of the technology. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

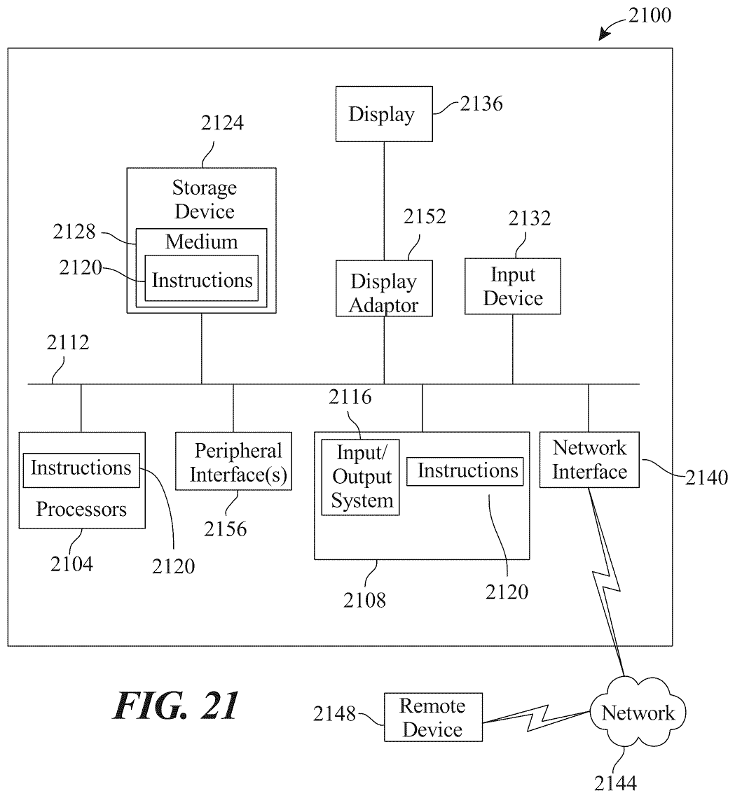

FIG. 21 shows a diagrammatic representation of one embodiment of a computing device in the exemplary form of a computer system 2100 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 2100 includes a processor 2104 and a memory 2108 that communicate with each other, and with other components, via a bus 2112. Bus 2112 may include any of several types of bus structures including, but not limited to, a memory bus, a memory controller, a peripheral bus, a local bus, and any combinations thereof, using any of a variety of bus architectures.

Processor 2104 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 2104 may be organized according to Von Neumann and/or Harvard architecture as a non-limiting example. Processor 2104 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 2108 may include various components (e.g., machine-readable media) including, but not limited to, a random-access memory component, a read only component, and any combinations thereof. In one example, a basic input/output system 2116 (BIOS), including basic routines that help to transfer information between elements within computer system 2100, such as during start-up, may be stored in memory 2108. Memory 2108 may also include (e.g., stored on one or more machine-readable media) instructions (e.g., software) 2120 embodying any one or more of the aspects and/or methodologies of the present disclosure. In another example, memory 2108 may further include any number of program modules including, but not limited to, an operating system, one or more application programs, other program modules, program data, and any combinations thereof.

Computer system 2100 may also include a storage device 2124. Examples of a storage device (e.g., storage device 2124) include, but are not limited to, a hard disk drive, a magnetic disk drive, an optical disc drive in combination with an optical medium, a solid-state memory device, and any combinations thereof. Storage device 2124 may be connected to bus 2112 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 2124 (or one or more components thereof) may be removably interfaced with computer system 2100 (e.g., via an external port connector (not shown)). Particularly, storage device 2124 and an associated machine-readable medium 2128 may provide nonvolatile and/or volatile storage of machine-readable instructions, data structures, program modules, and/or other data for computer system 2100. In one example, software 2120 may reside, completely or partially, within machine-readable medium 2128. In another example, software 2120 may reside, completely or partially, within processor 2104.

Computer system 2100 may also include an input device 2132. In one example, a user of computer system 2100 may enter commands and/or other information into computer system 2100 via input device 2132. Examples of an input device 2132 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 2132 may be interfaced to bus 2112 via any of a variety of interfaces (not shown) including, but not limited to, a serial interface, a parallel interface, a game port, a USB interface, a FIREWIRE interface, a direct interface to bus 2112, and any combinations thereof. Input device 2132 may include a touch screen interface that may be a part of or separate from display 2136, discussed further below. Input device 2132 may be utilized as a user selection device for selecting one or more graphical representations in a graphical interface as described above.

A user may also input commands and/or other information to computer system 2100 via storage device 2124 (e.g., a removable disk drive, a flash drive, etc.) and/or network interface device 2140. A network interface device, such as network interface device 2140, may be utilized for connecting computer system 2100 to one or more of a variety of networks, such as network 2144, and one or more remote devices 2148 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 2144, may employ a wired and/or a wireless mode of communication. In general, any network topology may be used. Information (e.g., data, software 2120, etc.) may be communicated to and/or from computer system 2100 via network interface device 2140.

Computer system 2100 may further include a video display adapter 2152 for communicating a displayable image to a display device, such as display device 2136. Examples of a display device include, but are not limited to, a liquid crystal display (LCD), a cathode ray tube (CRT), a plasma display, a light emitting diode (LED) display, and any combinations thereof. Display adapter 2152 and display device 2136 may be utilized in combination with processor 2104 to provide graphical representations of aspects of the present disclosure. In addition to a display device, computer system 2100 may include one or more other peripheral output devices including, but not limited to, an audio speaker, a printer, and any combinations thereof. Such peripheral output devices may be connected to bus 2112 via a peripheral interface 2156. Examples of a peripheral interface include, but are not limited to, a serial port, a USB connection, a FIREWIRE connection, a parallel connection, and any combinations thereof

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve compositions, systems, and methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A method of allergen testing, the method comprising:
obtaining a subject sample;
depleting IgG in the subject sample; and
determining a level of IgE specific to an allergen in the subject sample.

2. The method of claim 1, wherein depleting IgG in the subject sample comprises:
adding an IgG binder to the subject sample; and
adding an IgG binder aggregator to the subject sample.

3. The method of claim 2, wherein the IgG binder is bPG.

4. The method of claim 3, wherein the IgG binder aggregator is SA.

5. The method of claim 4, wherein bPG is added at a tIgG:PG molar ratio of 2.0 and SA is added at a PG: SA molar ratio of 2.0.

6. The method of claim 2, further comprising removing compounds from the subject sample as a function of their size.

7. The method of claim 6, wherein removing compounds from the subject sample as a function of their size comprises quick spin centrifugation.

8. The method of claim 6, wherein removing compounds from the subject sample as a function of their size comprises filtration using a 10 nm filter.

9. The method of claim 1, wherein the subject sample comprises subject blood, subject serum, or subject plasma.

10. The method of claim 1, wherein the allergen is an allergen from a source selected from the list consisting of alder (Alnus incana), birch (Betula alba/verrucosa), cypress (Cupressus sempervirens/arizonica), hazel (Corylus avellana), plane (Platanus vulgaris), grass mix (Poa pratensis, Dactilis glomerata, Lolium perenne, Phleum pratense, Festuca pratensis, Helictotrichon pratense), olive (Olea europea), mugwort (Artemisia vulgaris), ragweed (Ambrosia artemisiifolia), Alternaria alternata (tenuis), Cladosporium herbarum, Aspergillus fumigatus, Parietaria, cat (Felis domesticus), dog (Canis familiaris), dust mite (Dermatophagoides pteronyssinus/farinae), cockroach (Blatella germanica), milk, eggs, fish, crustacean shellfish, tree nuts, peanuts, wheat, and soybeans.

11. The method of claim 1, wherein determining the level of IgE specific to the allergen in the subject sample is done using a biolayer interferometry system.

12. The method of claim 11, wherein determining the level of IgE specific to the allergen in the subject sample is done using a biolayer interferometry system, wherein the biolayer interferometry system is configured to detect binding of a gold nanoparticle to a sensor.

13. The method of claim 12, wherein the gold nanoparticle is a 40 nm gold nanoparticle.

14. A method of IgG depletion, the method comprising:
obtaining a sample;
adding an IgG binder to the sample; and
adding an IgG binder aggregator to the sample.

15. The method of claim 14, wherein the IgG binder is bPG.

16. The method of claim 15, wherein the IgG binder aggregator is SA.

17. The method of claim 16, wherein bPG is added at a tIgG:PG molar ratio of 2.0 and SA is added at a PG: SA molar ratio of 2.0.

18. The method of claim 14, further comprising removing compounds from the sample as a function of their size.

19. The method of claim 18, wherein removing compounds from the subject sample as a function of their size comprises quick spin centrifugation.

20. The method of claim 18, wherein removing compounds from the subject sample as a function of their size comprises filtration using a 10 nm filter.
